(19) United States
(12) Patent Application Publication
(10) Pub. No.: US 2023/0295735 A1
(43) Pub. Date: Sep. 21, 2023

(54) SMARCA4 INHIBITION FOR THE TREATMENT OF CANCER

(71) Applicant: Epizyme, Inc., Cambridge, MA (US)

(72) Inventors: Allison E. DREW, Arlington, MA (US); Lindsey Wood EICHINGER, Watertown, MA (US)

(21) Appl. No.: 18/010,914

(22) PCT Filed: Jun. 17, 2021
(86) PCT No.: PCT/US2021/037849

§ 371 Date: Dec. 16, 2022

Related U.S. Application Data
(60) Provisional application No. 63/040,622, filed on Jun. 18, 2020.

(51) Int. Cl. C12Q 1/6886

(52) U.S. Cl. CPC C12Q 1/6886; C12Q 2600/106; C12Q 2600/158; C12Q 2600/136

(57) ABSTRACT

The present disclosure provides methods of determining a response to at least one therapy by a subject having cancer, wherein the at least one therapy comprises the administration of at least one SMARCA4-targeting compound. The present disclosure also provides methods of treating a cancer in a subject, wherein the subject has been previously administered at least one therapeutically effective amount of at least one SMARCA4-targeting compound. The present disclosure also provides a method of identifying at least one SMARCA4-targeting compound. The present disclosure also provides a method of modulating an epithelial/mesenchymal state in at least one cell.

### RELATED APPLICATIONS

This application claims priority to, and the benefit of, U.S. Provisional Application No. 63/040,622, filed Jun. 18, 2020, the content of which is incorporated herein by reference in its entirety.

### BACKGROUND

SMARCA4 is a SWI/SNF related, matrix associated, actin dependent regulator of chromatin. SMARCA4 is a subunit of the SWI/SNF complex, which regulates gene activity (expression) by a process known as chromatin remodeling. SWI/SNF complexes regulate many cell processes by direct modulation of nucleosomal structure. The catalytic subunit of SMARCA4 has ATP-dependent helicase activity that repositions nucleosomes. SMARCA4 and SMARCA2 are mutually exclusive paralogs in the SWI/SNF complex. SWI/SNF complex members are mutated in about 20% of human cancers. Accordingly, there is an unmet need in the art for methods of identifying SMARCA4-targeting compounds, methods of treating subjects using a SMARCA4-targeting compounds and methods to evaluate the response of such subjects to the administration of the SMARCA4-targeting compounds.

### SUMMARY

The present disclosure provides a method of determining a response to at least one therapy by a subject having a cancer, wherein the at least one therapy comprises the administration of at least one SMARCA4-targeting compound, the method comprising: a) determining a first expression level of at least one gene from at least one gene set in a biological sample collected from the subject at a first time point, wherein the first time point is prior to the administration of the at least one therapy; b) determining a second expression level of the least one gene in a biological sample collected from the subject at a second time point, wherein the second time point is after the administration of the at least one therapy; c) comparing the second expression level of the at least one gene to the first expression level of the at least one gene; and d) determining that the subject is responding to the at least one therapy when the second expression level of the at least one gene is greater than the first expression level of the at least one gene. In some embodiments of the preceding method, the at least one gene is selected from the group consisting of the genes recited in Table 1. In some embodiments of the preceding method, the at least one gene set is selected from the gene sets recited in Table 2.

In some embodiments of the preceding method, step (d) comprises determining that the subject is responding to the at least one therapy when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the first expression level of the at least one gene.

The present disclosure provides a method of treating a cancer in a subject, the method comprising: a) determining a first expression level of at least one gene from at least one gene set in a biological sample collected from the subject at a first time point, wherein the first time point is prior to the administration of at least one therapeutically effective amount of at least one SMARCA4-targeting compound; b) determining a second expression level of the least one gene in a biological sample collected from the subject at a second time point, wherein the second time point is after the administration of at least one therapeutically effective amount of at least one SMARCA4-targeting compound; c) comparing the second expression level of the at least one gene to the first expression level of the at least one gene; and d) administering to the subject at least one additional therapeutically effective amount of the at least one SMARCA4-targeting compound when the second expression level of the at least one gene is greater than the first expression level of the at least one gene, or administering at least one alternative therapy to the subject when the second expression level of the at least one gene is less than the first expression level of the at least one gene. In some embodiments of the preceding method, the at least one gene is selected from the group consisting of the genes recited in Table 1. In some embodiments of the preceding method, the at least one gene set is selected from the gene sets recited in Table 2.

In some embodiments of the preceding method, step (d) comprises administering to the subject at least one additional therapeutically effective amount of the at least one SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, greater than the first expression level of the at least one gene, or else administering at least one alternative therapy to the subject.

The present disclosure provides a method of determining a response to at least one therapy by a subject having a cancer, wherein the at least one therapy comprises the administration of at least one SMARCA4-targeting compound, the method comprising: a) determining the expression level of at least one gene from at least one gene set in a biological sample collected from the subject at a first time point, wherein the first time point is after the administration of the at least one therapy; b) comparing the expression level of the at least one gene to at least one corresponding predetermined cutoff value; and c) determining that the subject is responding to the at least one therapy when the expression level of the at least one gene is greater than the at least one corresponding predetermined cutoff value. In some embodiments of the preceding method, the at least one gene is selected from the group consisting of the genes recited in Table 1. In some embodiments of the preceding method, the at least one gene set is selected from the gene sets recited in Table 2.

In some embodiments of the preceding method, step (c) comprises determining that the subject is responding to the at least one therapy when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the at least one corresponding predetermined cutoff value.

The present disclosure provides a method of treating a cancer in a subject, wherein the subject has been previously administered at least one therapeutically effective amount of at least one SMARCA4-targeting compound, the method comprising: a) determining the expression level of at least one gene from at least one gene set in a biological sample from the subject; b) comparing the expression level of the at least one gene to at least one corresponding predetermined cutoff value; and c) administering to the subject at least one additional therapeutically effective amount of the at least one SMARCA4-targeting compound when the expression level of the at least one gene is greater than the at least one corresponding predetermined cutoff value, or administering at least one alternative therapy to the subject when the expression level of the at least one gene is less than the at least one corresponding predetermined cutoff value. In some embodiments of the preceding method, the at least one gene is selected from the group consisting of the genes recited in Table 1. In some embodiments of the preceding method, the at least one gene set is selected from the gene sets recited in Table 2.

In some embodiments of the preceding method, step (c) comprises administering to the subject at least one additional therapeutically effective amount of the at least one SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the at least one corresponding predetermined cutoff value, or else administering at least one alternative therapy to the subject.

The present disclosure provides a method of determining a response to at least one therapy by a subject having a cancer, wherein the at least one therapy comprises the administration of at least one SMARCA4-targeting compound, the method comprising: a) determining a first expression level of at least one gene from at least one gene set in a biological sample collected from the subject at a first time point, wherein the first time point is prior to the administration of the at least one therapy; b) determining a second expression level of the least one gene in a biological sample collected from the subject at a second time point, wherein the second time point is after the administration of the at least one therapy; c) comparing the second expression level of the at least one gene to the first expression level of the at least one gene; and d) determining that the subject is responding to the at least one therapy when the second expression level of the at least one gene is less than the first expression level of the at least one gene. In some embodiments of the preceding method, the least one gene is selected from the group consisting of the genes recited in Table 3. In some embodiments of the preceding method, the at least one gene set is selected from the gene sets recited in Table 4.

In some embodiments of the preceding method, step (d) comprises determining that the subject is responding to the at least one therapy when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the first expression level of the at least one gene.

The present disclosure provides a method of treating a cancer in a subject, the method comprising: a) determining a first expression level of at least one gene from at least one gene set in a biological sample collected from the subject at a first time point, wherein the first time point is prior to the administration of at least one therapeutically effective amount of at least one SMARCA4-targeting compound; b) determining a second expression level of the least one gene in a biological sample collected from the subject at a second time point, wherein the second time point is after the administration of at least one therapeutically effective amount of at least one SMARCA4-targeting compound; c) comparing the second expression level of the at least one gene to the first expression level of the at least one gene; and d) administering to the subject at least one additional therapeutically effective amount of the at least one SMARCA4-targeting compound when the second expression level of the at least one gene is less than the first expression level of the at least one gene, or administering at least one alternative therapy to the subject when the second expression level of the at least one gene is greater than the first expression level of the at least one gene. In some embodiments of the preceding method, the least one gene is selected from the group consisting of the genes recited in Table 3. In some embodiments of the preceding method, the at least one gene set is selected from the gene sets recited in Table 4.

In some embodiments of the preceding method, step (d) comprises administering to the subject at least one additional therapeutically effective amount of the at least one SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the first expression level of the at least one gene, or else administering at least one alternative therapy to the subject.

The present disclosure provides a method of determining a response to at least one therapy by a subject having a cancer, wherein the at least one therapy comprises the administration of at least one SMARCA4-targeting compound, the method comprising: a) determining the expression level of at least one gene from at least one gene set in a biological sample collected from the subject at a first time point, wherein the first time point is after the administration of the at least one therapy; b) comparing the expression level of the at least one gene to at least one corresponding predetermined cutoff value; and c) determining that the subject is responding to the at least one therapy when the expression level of the at least one gene is less than the at least one corresponding predetermined cutoff value. In some embodiments of the preceding method, the least one gene is selected from the group consisting of the genes recited in Table 3. In some embodiments of the preceding method, the at least one gene set is selected from the gene sets recited in Table 4.

In some embodiments of the preceding method, step (c) comprises determining that the subject is responding to the at least one therapy when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the at least one corresponding predetermined cutoff value.

The present disclosure provides a method of treating a cancer in a subject, wherein the subject has been previously administered at least one therapeutically effective amount of at least one SMARCA4-targeting compound, the method comprising: a) determining the expression level of at least one gene from at least one gene set in a biological sample from the subject; b) comparing the expression level of the at least one gene to at least one corresponding predetermined cutoff value; and c) administering to the subject at least one additional therapeutically effective amount of the at least one SMARCA4-targeting compound when the expression level of the at least one gene is less than the at least one corresponding predetermined cutoff value, or administering at least one alternative therapy to the subject when the expression level of the at least one gene is greater than the at least one corresponding predetermined cutoff value. In some embodiments of the preceding method, the least one gene is selected from the group consisting of the genes recited in Table 3. In some embodiments of the preceding method, the at least one gene set is selected from the gene sets recited in Table 4.

In some embodiments of the preceding method, step (c) comprises administering to the subject at least one additional therapeutically effective amount of the at least one SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the at least one corresponding predetermined cutoff value, or else administering at least one alternative therapy to the subject.

The present disclosure provides a method of identifying at least one SMARCA4-targeting compound, the method comprising: a) determining a first expression level of at least one gene from at least one gene set in a plurality of cells at a first time point, wherein the plurality of cells exhibits aberrant SMARCA2 expression, activity or a combination thereof; b) treating the plurality of cells with at least one amount of at least one test compound; c) determining a second expression level of the least one gene in the plurality of cells at a second time point; d) comparing the second expression level of the at least one gene to the first expression level of the at least one gene; and e) identifying the at least one test compound as a SMARCA4-targeting compound when the second expression level of the at least one gene is greater than the first expression level of the at least one gene. In some embodiments of the preceding method, the at least one gene is selected from the group consisting of the genes recited in Table 1. In some embodiments of the preceding method, the at least one gene set is selected from the gene sets recited in Table 2.

In some embodiments of the preceding method, step (e) comprises identifying the at least one test compound as a SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the first expression level of the at least one gene.

The present disclosure provides a method of identifying at least one SMARCA4-targeting compound, the method comprising: a) treating at least one cell with at least one amount of at least one test compound, wherein the at least one cell exhibits aberrant SMARCA2 expression, activity or a combination thereof; b) determining the expression level of at least one gene from at least one gene set in the at least one cell; c) comparing the expression level of the at least one gene to at least one corresponding predetermined cutoff value; and d) identifying the at least one test compound as a SMARCA4-targeting compound when the expression level of the at least one gene is greater than the at least one corresponding predetermined cutoff value. In some embodiments of the preceding method, the at least one gene is selected from the group consisting of the genes recited in Table 1. In some embodiments of the preceding method, the at least one gene set is selected from the gene sets recited in Table 2.

In some embodiments of the preceding method, step (d) comprises identifying the at least one test compound as a SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the at least one corresponding predetermined cutoff value.

The present disclosure provides a method of identifying at least one SMARCA4-targeting compound, the method comprising: a) determining a first expression level of at least one gene from at least one gene set in a plurality of cells at a first time point, wherein the plurality of cells exhibits aberrant SMARCA2 expression, activity or a combination thereof; b) treating the plurality of cells with at least one amount of at least one test compound; c) determining a second expression level of the least one gene in the plurality of treated cells at a second time point; d) comparing the second expression level of the at least one gene to the first expression level of the at least one gene; and e) identifying the at least one test compound as a SMARCA4-targeting compound when the second expression level of the at least one gene is less than the first expression level of the at least one gene. In some embodiments of the preceding method, the least one gene is selected from the group consisting of the genes recited in Table 3. In some embodiments of the preceding method, the at least one gene set is selected from the gene sets recited in Table 4.

In some embodiments of the preceding method, step (e) comprises identifying the at least one test compound as a SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the first expression level of the at least one gene.

The present disclosure provides a method of identifying at least one SMARCA4-targeting compound, the method comprising: a) treating at least one cell with at least one amount of at least one test compound, wherein the at least one cell exhibits aberrant SMARCA2 expression, activity or a combination thereof; b) determining the expression level of at least one gene from at least one gene set in the at least treated one cell; c) comparing the expression level of the at least one gene to at least one corresponding predetermined cutoff value; and d) identifying the at least one test compound as a SMARCA4-targeting compound when the expression level of the at least one gene is less than the at least one corresponding predetermined cutoff value. In some embodiments of the preceding method, the least one gene is selected from the group consisting of the genes recited in Table 3. In some embodiments of the preceding method, the at least one gene set is selected from the gene sets recited in Table 4.

In some embodiments of the preceding method, step (d) comprises identifying the at least one test compound as a SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the at least one corresponding predetermined cutoff value.

In some embodiments of the preceding methods, the cancer exhibits aberrant SMARCA2 expression, activity, function or a combination thereof.

In some embodiments, aberrant SMARCA2 expression comprises decreased SMARCA2 expression as compared to a control expression level. In some embodiments, the control expression level is the expression level of SMARCA2 in a subject that does not have cancer.

In some embodiments, aberrant SMARCA2 activity comprises decreased SMARCA2 activity as compared to a control activity level. In some embodiments, the control activity level is the activity level of SMARCA2 in a subject that does not have cancer.

In some embodiments of the preceding methods, the at least one SMARCA4-targeting compound is a SMARCA4 inhibitor.

The present disclosure provides a method of modulating an epithelial/mesenchymal state in at least one cell comprising contacting the at least one cell with an effective amount of at least one SMARCA4-targeting compound. In some embodiments, the SMARCA4-targeting compound is a SMARCA4 inhibitor.

In some embodiments of the preceding methods, the cell is a cancer cell.

In some embodiments of the preceding methods, the cell exhibits aberrant SMARCA2 expression, activity or a combination thereof.

In some embodiments of the preceding methods, the cell exhibits aberrant SMARCA4 expression, activity or a combination thereof.

In some embodiments of the preceding methods, modulating an epithelial/mesenchymal state in the at least one cell comprises altering the expression level of at least one gene and/or protein associated with an epithelial state. In some embodiments, the at least one gene and/or protein associated with an epithelial state is E-cadherin, FOXA1 or CLDN1.

In some embodiments of the preceding methods, modulating an epithelial/mesenchymal state in the at least one cell comprises altering the expression level of at least one gene and/or protein associated with a mesenchymal state. In some embodiments, the at least one gene and/or protein associated with a mesenchymal state is N-cadherin, vimentin, SNAI1 or ZEB1.

Any of the above aspects can be combined with any other aspect.

Unless otherwise defined, all technical and scientific terms used herein have the same meaning as commonly understood by one of ordinary skill in the art to which this disclosure belongs. In the specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.019% of the stated value. Unless otherwise clear from the context, all numerical values provided herein are modified by the term “about.” Unless specifically stated or obvious from context, as used herein, the term “or” is understood to be inclusive and covers both “or” and “and”.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a series of charts showing principal component analysis of transcriptional changes (left) and changes in expression levels of specific genes (right) in H358 cells (Parental), SMARCA2-knockout H358 cells (SMARCA2 KO; S2-B3 and S2-C2), and SMARCA4-knockout H358 cells (SMARCA4 KO; S4-D8 and S4-E4) upon treatment with a SMARCA4-targeting compound (1 μM or 10 μM) or a DMSO vehicle control. The individual genes shown in the graphs on the right are examples of genes whose expression changes are weighted heavily in the principal components indicated.

FIG. 2 is a series of charts showing the expression level of TP63 (upper chart) and FOXA1 (lower chart) in H358 cells, SMARCA2-knockout H358 cells (S2-B3 and S2-C2), and SMARCA4-knockout H358 cells (S4-D8 and S4-E4) upon treatment with a SMARCA4-targeting compound (1 μM or 10 μM) or a DMSO vehicle control.

FIG. 3 is a chart showing the expression level of CDH1 in H358 cells, SMARCA2-knockout H358 cells (S2-B3 and S2-C2), and SMARCA4-knockout H358 cells (S4-D8 and S4-E4) upon treatment with a SMARCA4-targeting compound (1 μM or 10 μM) or a DMSO vehicle control.

FIG. 4 is a series of charts showing the expression level of SNAI1 (left) and ZEB1 (right) in H358 cells, SMARCA2-knockout H358 cells (S2-B3 and S2-C2), and SMARCA4-knockout H358 cells (S4-D8 and S4-E4) upon treatment with a SMARCA4-targeting compound (1 μM or 10 μM) or a DMSO vehicle control.

FIG. 5 is a series of charts showing the expression level of E-cadherin (upper chart) and CLDN1 (lower chart) in H358 cells, SMARCA2-knockout H358 cells (S2-B3 and S2-C2), and SMARCA4-knockout H358 cells (S4-D8 and S4-E4) upon treatment with a SMARCA4-targeting compound (0.1 μM, 1 μM or 10 μM) or a DMSO vehicle control. The insets show the expression of E-cadherin and CLDN1 in H358 cells upon treatment with DMSO or StemXVivo EMT Inducing Media Supplement (R&D Systems).

FIG. 6 is a series of charts showing the expression level of vimentin (upper chart) and N-cadherin (lower chart) in H358 cells, SMARCA2-knockout H358 cells (S2-B3 and S2-C2), and SMARCA4-knockout H358 cells (S4-D8 and S4-E4) upon treatment with a SMARCA4-targeting compound (0.1 μM, 1 μM or 10 μM) or a DMSO vehicle control. The inserts show the expression of vimentin and N-cadherin in H358 cells upon treatment with DMSO or StemXVivo EMT Inducing Media Supplement (R&D Systems).

### DETAILED DESCRIPTION

The present disclosure provides methods of determining a response to at least one therapy by a subject having cancer, wherein the at least one therapy comprises the administration of at least one SMARCA4-targeting compound, the method comprising determining the expression level of at least one gene from at least one gene set described herein. The present disclosure also provides methods of treating a cancer in a subject, wherein the subject has been previously administered at least one therapeutically effective amount of at least one SMARCA4-targeting compound, the method comprising determining the expression level of at least one gene from at least one gene set described herein. The present disclosure also provides a method of identifying at least one SMARCA4-targeting compound, the method comprising determining the expression level of at least one gene from at least one gene set described herein. The present disclosure also provides a method of modulating an epithelial/mesenchymal state in at least one cell, the method comprising contacting the at least one cell with an effective amount of a compound that targets SMARCA4. In some embodiments, the SMARCA4-targeting compound may also target or inhibit other genes, for example, SMARCA2.

In some aspects, the present disclosure provides a method of determining a response to at least one therapy by a subject having a cancer, wherein the at least one therapy comprises the administration of at least one SMARCA4-targeting compound, the method comprising: a) determining the expression level of at least one gene from at least one gene set in a biological sample collected from the subject at a first time point, wherein the first time point is after the administration of the at least one therapy; b) comparing the expression level of the at least one gene to at least one corresponding predetermined cutoff value; and c) determining that the subject is responding to the at least one therapy when the expression level of the at least one gene is greater than the at least one corresponding predetermined cutoff value. In some embodiments of the preceding method, the at least one gene is selected from the group consisting of the genes recited in Table 1. In some embodiments of the preceding method, the at least one gene set is selected from the gene sets recited in Table 2.

In some aspects, the present disclosure provides a method of determining a response to at least one therapy by a subject having a cancer, wherein the at least one therapy comprises the administration of at least one SMARCA4-targeting compound, the method comprising: a) determining the expression level of at least one gene from at least one gene set in a biological sample collected from the subject at a first time point, wherein the first time point is after the administration of the at least one therapy; b) comparing the expression level of the at least one gene to at least one corresponding predetermined cutoff value; and c) determining that the subject is responding to the at least one therapy when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times greater than the at least one corresponding predetermined cutoff value. In some embodiments of the preceding method, the at least one gene is selected from the group consisting of the genes recited in Table 1 which includes genes that are upregulated in SMARCA2-knockout cell lines upon treatment with a SMARCA4-targeting compound. In some embodiments of the preceding method, the at least one gene set is selected from the gene sets recited in Table 2, which are upregulated in SMARCA2-knockout cell lines upon treatment with a SMARCA4-targeting compound.

TABLE 1

| | | | | |
|---|---|---|---|---|
| ABHD12 | TOM1L2 | BCAM | ALDH3B1 | ESPN |
| TPP1 | DMPK | CUL7 | CRIP1 | FLVCR2 |
| PLS3 | SLC44A2 | LMO7 | GPRC5A | KCNK6 |
| ARF6 | ERBB2 | FAM129B | ILIR1 | VSIR |
| GNAQ | CAST | USP54 | TMPRSS11E | VIPR1 |
| TMEM120A | RELB | LRP11 | PADI2 | SYNPO |
| B4GALT4 | TPM1 | EVPL | PAQR7 | UPK2 |
| IL17RC | MYOF | IFITM10 | PPL | CLIC3 |
| ANXA1 | TRPM4 | KANK2 | PORCN | SERHL2 |
| HYAL2 | CYHR1 | INPP4A | GSN | SUSD2 |
| S100A11 | ASMTL | NTN1 | VSIG10 | BNIPL |
| CTSA | GAB1 | LIPH | LYPD3 | KRT80 |
| SYP | NR4A3 | NR4A2 | | |

TABLE 2
Gene Set
HALLMARK_TGF_BETA_SIGNALING

In some aspects, the present disclosure provides a method of determining a response to at least one therapy by a subject having a cancer, wherein the at least one therapy comprises the administration of at least one SMARCA4-targeting compound, the method comprising: a) determining the expression level of the genes from at least one gene set in a biological sample collected from the subject at a first time point, wherein the first time point is after the administration of the at least one therapy; b) determining whether the at least one gene set is upregulated in the biological sample as compared to a reference sample, based on the expression levels measured in step (a); and c) determining that the subject is responding to the at least one therapy when the at least one gene set is upregulated in the biological sample as compared to the reference sample.

In some aspects, the present disclosure provides a method of determining a response to at least one therapy by a subject having a cancer, wherein the at least one therapy comprises the administration of at least one SMARCA4-targeting compound, the method comprising: a) determining the expression level of at least one gene from at least one gene set in a biological sample collected from the subject at a first time point, wherein the first time point is after the administration of the at least one therapy; b) comparing the expression level of the at least one gene to at least one corresponding predetermined cutoff value; and c) determining that the subject is responding to the at least one therapy when the expression level of the at least one gene is less than the at least one corresponding predetermined cutoff value. In some embodiments of the preceding method, the at least one gene is selected from the group consisting of the genes recited in Table 3 which includes genes which are downregulated in SMARCA2-knockout cell lines upon treatment with a SMARCA4-targeting compound. In some embodiments of the preceding method, the at least one gene set is selected from the gene sets recited in Table 4, which include gene sets downregulated in SMARCA2-knockout cell lines upon treatment with a SMARCA4-targeting compound.

TABLE 3

| | | | |
|---|---|---|---|
| ADGRF5 | GCLM | MTHFD2 | MCM8 |
| CFI | GPRC5C | CBX2 | CTNNB1 |
| MMP13 | ALCAM | CDCA7 | BLM |
| TGFBI | STK39 | KDSR | GGCT |
| HEG1 | TUBE1 | CONB1IP1 | FANCD2 |
| RAB38 | ATP1B1 | MPHOSPH9 | SLC1A5 |
| BCAT1 | HIST1H3I | TBL1XRI | CPD |
| LPIN2 | VEGFA | RAD54L | POC1B |
| ASNS | ZNF318 | MCM6 | PSPH |
| ETV1 | AJUBA | SPRED1 | FOXA2 |
| STS | S100P | ARHGDIB | |

TABLE 4
Gene Set
HALLMARK_E2F_TARGETS
HALLMARK_G2M_CHECKPOINT
HALLMARK_MYC_TARGETS_V1
HALLMARK_MTORC1_SIGNALING
HALLMARK_INTERFERON_ALPHA_RESPONSE
HALLMARK_MYC_TARGETS_V2
HALLMARK_INTERFERON_GAMMA_RESPONSE

In some aspects, the present disclosure provides a method of determining a response to at least one therapy by a subject having a cancer, wherein the at least one therapy comprises the administration of at least one SMARCA4-targeting compound, the method comprising: a) determining the expression level of at least one gene from at least one gene set in a biological sample collected from the subject at a first time point, wherein the first time point is after the administration of the at least one therapy; b) comparing the expression level of the at least one gene to at least one corresponding predetermined cutoff value; and c) determining that the subject is responding to the at least one therapy when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times less than the at least one corresponding predetermined cutoff value. In some embodiments of the preceding method, the at least one gene is selected from the group consisting of the genes recited in Table 3. In some embodiments of the preceding method, the at least one gene set is selected from the gene sets recited in Table 4.

In some aspects, the present disclosure provides a method of determining a response to at least one therapy by a subject having a cancer, wherein the at least one therapy comprises the administration of at least one SMARCA4-targeting compound, the method comprising: a) determining the expression level of the genes from at least one gene set in a biological sample collected from the subject at a first time point, wherein the first time point is after the administration of the at least one therapy; b) determining whether the at least one gene set is downregulated in the biological sample as compared to a reference sample, based on the expression levels measured in step (a); and c) determining that the subject is responding to the at least one therapy when the at least one gene set is downregulated in the biological sample as compared to the reference sample.

In some aspects, the present disclosure provides a method of treating a cancer in a subject, wherein the subject has been previously administered at least one therapeutically effective amount of at least one SMARCA4-targeting compound, the method comprising: a) determining the expression level of at least one gene from at least one gene set in a biological sample from the subject; b) comparing the expression level of the at least one gene to at least one corresponding predetermined cutoff value; and c) administering to the subject at least one additional therapeutically effective amount of the at least one SMARCA4-targeting compound when the expression level of the at least one gene is greater than the at least one corresponding predetermined cutoff value, or administering at least one alternative therapy to the subject when the expression level of the at least one gene is less than the at least one corresponding predetermined cutoff value. In some embodiments of the preceding method, the at least one gene is selected from the group consisting of the genes recited in Table 1. In some embodiments of the preceding method, the at least one gene set is selected from the gene sets recited in Table 2.

In some aspects, the present disclosure provides a method of treating a cancer in a subject, wherein the subject has been previously administered at least one therapeutically effective amount of at least one SMARCA4-targeting compound, the method comprising: a) determining the expression level of at least one gene from at least one gene set in a biological sample from the subject; b) comparing the expression level of the at least one gene to at least one corresponding predetermined cutoff value; and c) administering to the subject at least one additional therapeutically effective amount of the at least one SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times greater than the at least one corresponding predetermined cutoff value, or else administering at least one alternative therapy to the subject. In some embodiments of the preceding method, the at least one gene is selected from the group consisting of the genes recited in Table 1. In some embodiments of the preceding method, the at least one gene set is selected from the gene sets recited in Table 2.

In some aspects, the present disclosure provides a method of treating a cancer in a subject, wherein the subject has been previously administered at least one therapeutically effective amount of at least one SMARCA4-targeting compound, the method comprising: a) determining the expression level of the genes from at least one gene set in a biological sample collected from the subject at a first time point, wherein the first time point is after the administration of the at least one therapeutically effective amount of at least one SMARCA4-targeting compound; b) determining whether the at least one gene set is upregulated in the biological sample as compared to a reference sample, based on the expression levels measured in step (a); and; and c) administering to the subject at least one additional therapeutically effective amount of the at least one SMARCA4-targeting compound when the at least one gene set is upregulated in the biological sample as compared to the reference sample, or else administering at least one alternative therapy to the subject when the at least one gene set is not upregulated in the biological sample as compared to the reference sample.

In some aspects, the present disclosure provides a method of treating a cancer in a subject, wherein the subject has been previously administered at least one therapeutically effective amount of at least one SMARCA4-targeting compound, the method comprising: a) determining the expression level of at least one gene from at least one gene set in a biological sample from the subject; b) comparing the expression level of the at least one gene to at least one corresponding predetermined cutoff value; and c) administering to the subject at least one additional therapeutically effective amount of the at least one SMARCA4-targeting compound when the expression level of the at least one gene is less than the at least one corresponding predetermined cutoff value, or administering at least one alternative therapy to the subject when the expression level of the at least one gene is greater than the at least one corresponding predetermined cutoff value. In some embodiments of the preceding method, the at least one gene is selected from the group consisting of the genes recited in Table 3. In some embodiments of the preceding method, the at least one gene set is selected from the gene sets recited in Table 4.

In some aspects, the present disclosure provides a method of treating a cancer in a subject, wherein the subject has been previously administered at least one therapeutically effective amount of at least one SMARCA4-targeting compound, the method comprising: a) determining the expression level of at least one gene from at least one gene set in a biological sample from the subject; b) comparing the expression level of the at least one gene to at least one corresponding predetermined cutoff value; and c) administering to the subject at least one additional therapeutically effective amount of the at least one SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times less than the at least one corresponding predetermined cutoff value, or else administering at least one alternative therapy to the subject. In some embodiments of the preceding method, the at least one gene is selected from the group consisting of the genes recited in Table 3. In some embodiments of the preceding method, the at least one gene set is selected from the gene sets recited in Table 4.

In some aspects, the present disclosure provides a method of treating a cancer in a subject, wherein the subject has been previously administered at least one therapeutically effective amount of at least one SMARCA4-targeting compound, the method comprising: a) determining the expression level of the genes from at least one gene set in a biological sample collected from the subject at a first time point, wherein the first time point is after the administration of the at least one therapeutically effective amount of at least one SMARCA4-targeting compound; b) determining whether the at least one gene set is downregulated in the biological sample as compared to a reference sample, based on the expression levels measured in step (a); and; and c) administering to the subject at least one additional therapeutically effective amount of the at least one SMARCA4-targeting compound when the gene set is downregulated in the biological sample as compared to the reference sample, or else administering at least one alternative therapy to the subject when the at least one gene set is not downregulated in the biological sample as compared to the reference sample.

In some aspects, the present disclosure provides a method of determining a response to at least one therapy by a subject having a cancer, wherein the at least one therapy comprises the administration of at least one SMARCA4-targeting compound, the method comprising: a) determining a first expression level of at least one gene from at least one gene set in a biological sample collected from the subject at a first time point, wherein the first time point is prior to the administration of the at least one therapy; b) determining a second expression level of the least one gene in a biological sample collected from the subject at a second time point, wherein the second time point is after the administration of the at least one therapy; c) comparing the second expression level of the at least one gene to the first expression level of the at least one gene; and d) determining that the subject is responding to the at least one therapy when the second expression level of the at least one gene is greater than the first expression level of the at least one gene. In some embodiments of the preceding method, the at least one gene is selected from the group consisting of the genes recited in Table 1. In some embodiments of the preceding method, the at least one gene set is selected from the gene sets recited in Table 2.

In some aspects, the present disclosure provides a method of determining a response to at least one therapy by a subject having a cancer, wherein the at least one therapy comprises the administration of at least one SMARCA4-targeting compound, the method comprising: a) determining a first expression level of at least one gene from at least one gene set in a biological sample collected from the subject at a first time point, wherein the first time point is prior to the administration of the at least one therapy; b) determining a second expression level of the least one gene in a biological sample collected from the subject at a second time point, wherein the second time point is after the administration of the at least one therapy; c) comparing the second expression level of the at least one gene to the first expression level of the at least one gene; and d) determining that the subject is responding to the at least one therapy when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times greater than the first expression level of the at least one gene. In some embodiments of the preceding method, the at least one gene is selected from the group consisting of the genes recited in Table 1. In some embodiments of the preceding method, the at least one gene set is selected from the gene sets recited in Table 2.

In some aspects, the present disclosure provides a method of determining a response to at least one therapy by a subject having a cancer, wherein the at least one therapy comprises the administration of at least one SMARCA4-targeting compound, the method comprising: a) determining a first expression level of the genes from at least one gene set in a biological sample collected from the subject at a first time point, wherein the first time point is prior to the administration of the at least one therapy; b) determining a second expression level of the genes from the at least one gene set in a biological sample collected from the subject at a second time point, wherein the second time point is after the administration of the at least one therapy; c) determining whether the at least one gene set is upregulated in the biological sample collected from the subject at the second time point as compared to the biological sample collected from the subject at the first time point, based on the expression levels measured in steps (a) and (b); and d) determining that the subject is responding to the at least one therapy when the at least one gene set is upregulated in the biological sample collected from the subject at the second time point as compared to the biological sample collected from the subject at the first time point.

In some aspects, the present disclosure provides a method of determining a response to at least one therapy by a subject having a cancer, wherein the at least one therapy comprises the administration of at least one SMARCA4-targeting compound, the method comprising: a) determining a first expression level of at least one gene from at least one gene set in a biological sample collected from the subject at a first time point, wherein the first time point is prior to the administration of the at least one therapy; b) determining a second expression level of the least one gene in a biological sample collected from the subject at a second time point, wherein the second time point is after the administration of the at least one therapy; c) comparing the second expression level of the at least one gene to the first expression level of the at least one gene; and d) determining that the subject is responding to the at least one therapy when the second expression level of the at least one gene is less than the first expression level of the at least one gene. In some embodiments of the preceding method, the at least one gene is selected from the group consisting of the genes recited in Table 3. In some embodiments of the preceding method, the at least one gene set is selected from the gene sets recited in Table 4.

In some aspects, the present disclosure provides a method of determining a response to at least one therapy by a subject having a cancer, wherein the at least one therapy comprises the administration of at least one SMARCA4-targeting compound, the method comprising: a) determining a first expression level of at least one gene from at least one gene set in a biological sample collected from the subject at a first time point, wherein the first time point is prior to the administration of the at least one therapy; b) determining a second expression level of the least one gene in a biological sample collected from the subject at a second time point, wherein the second time point is after the administration of the at least one therapy; c) comparing the second expression level of the at least one gene to the first expression level of the at least one gene; and d) determining that the subject is responding to the at least one therapy when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times less than the first expression level of the at least one gene. In some embodiments of the preceding method, the at least one gene is selected from the group consisting of the genes recited in Table 3. In some embodiments of the preceding method, the at least one gene set is selected from the gene sets recited in Table 4.

In some aspects, the present disclosure provides a method of determining a response to at least one therapy by a subject having a cancer, wherein the at least one therapy comprises the administration of at least one SMARCA4-targeting compound, the method comprising: a) determining a first expression level of the genes from at least one gene set in a biological sample collected from the subject at a first time point, wherein the first time point is prior to the administration of the at least one therapy; b) determining a second expression level of the genes from the at least one gene set in a biological sample collected from the subject at a second time point, wherein the second time point is after the administration of the at least one therapy; c) determining whether the at least one gene set is downregulated in the biological sample collected from the subject at the second time point as compared to the biological sample collected from the subject at the first time point, based on the expression levels measured in steps (a) and (b); and d) determining that the subject is responding to the at least one therapy when the at least one gene set is downregulated in the biological sample collected from the subject at the second time point as compared to the biological sample collected from the subject at the first time point.

In some aspects, the present disclosure provides a method of treating a cancer in a subject, the method comprising: a) determining a first expression level of at least one gene from at least one gene set in a biological sample collected from the subject at a first time point, wherein the first time point is prior to the administration of at least one therapeutically effective amount of at least one SMARCA4-targeting compound; b) determining a second expression level of the least one gene in a biological sample collected from the subject at a second time point, wherein the second time point is after the administration of at least one therapeutically effective amount of at least one SMARCA4-targeting compound; c) comparing the second expression level of the at least one gene to the first expression level of the at least one gene; and d) administering to the subject at least one additional therapeutically effective amount of the at least one SMARCA4-targeting compound when the second expression level of the at least one gene is greater than the first expression level of the at least one gene, or administering at least one alternative therapy to the subject when the second expression level of the at least one gene is less than the first expression level of the at least one gene. In some embodiments of the preceding method, the at least one gene is selected from the group consisting of the genes recited in Table 1. In some embodiments of the preceding method, the at least one gene set is selected from the gene sets recited in Table 2.

In some aspects, the present disclosure provides a method of treating a cancer in a subject, the method comprising: a) determining a first expression level of at least one gene from at least one gene set in a biological sample collected from the subject at a first time point, wherein the first time point is prior to the administration of at least one therapeutically effective amount of at least one SMARCA4-targeting compound; b) determining a second expression level of the least one gene in a biological sample collected from the subject at a second time point, wherein the second time point is after the administration of at least one therapeutically effective amount of at least one SMARCA4-targeting compound; c) comparing the second expression level of the at least one gene to the first expression level of the at least one gene; and d) administering to the subject at least one additional therapeutically effective amount of the at least one SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times greater than the first expression level of the at least one gene, or else administering at least one alternative therapy to the subject. In some embodiments of the preceding method, the at least one gene is selected from the group consisting of the genes recited in Table 1. In some embodiments of the preceding method, the at least one gene set is selected from the gene sets recited in Table 2.

In some aspects, the present disclosure provides a method of treating a cancer in a subject, the method comprising: a) determining a first expression level of the genes from at least one gene set in a biological sample collected from the subject at a first time point, wherein the first time point is prior to the administration of at least one therapeutically effective amount of at least one SMARCA4-targeting compound; b) determining a second expression level of the genes from the at least one gene set in a biological sample collected from the subject at a second time point, wherein the second time point is after the administration of at least one therapeutically effective amount of at least one SMARCA4-targeting compound; c) determining whether the at least one gene set is upregulated in the biological sample collected from the subject at the second time point as compared to the biological sample collected from the subject at the first time point, based on the expression levels measured in steps (a) and (b); and d) administering to the subject at least one additional therapeutically effective amount of the at least one SMARCA4-targeting compound when the at least one gene set is upregulated in the biological sample collected from the subject at the second time point as compared to the biological sample collected from the subject at the first time point, or else administering at least one alternative therapy to the subject when the at least one gene set is not upregulated in the biological sample collected from the subject at the second time point as compared to the biological sample collected from the subject at the first time point.

In some aspects, the present disclosure provides a method of treating a cancer in a subject, the method comprising: a) determining a first expression level of at least one gene from at least one gene set in a biological sample collected from the subject at a first time point, wherein the first time point is prior to the administration of at least one therapeutically effective amount of at least one SMARCA4-targeting compound; b) determining a second expression level of the least one gene in a biological sample collected from the subject at a second time point, wherein the second time point is after the administration of at least one therapeutically effective amount of at least one SMARCA4-targeting compound; c) comparing the second expression level of the at least one gene to the first expression level of the at least one gene; and d) administering to the subject at least one additional therapeutically effective amount of the at least one SMARCA4-targeting compound when the second expression level of the at least one gene is less than the first expression level of the at least one gene, or administering at least one alternative therapy to the subject when the second expression level of the at least one gene is greater than the first expression level of the at least one gene. In some embodiments of the preceding method, the at least one gene is selected from the group consisting of the genes recited in Table 3. In some embodiments of the preceding method, the at least one gene set is selected from the gene sets recited in Table 4.

In some aspects, the present disclosure provides a method of treating a cancer in a subject, the method comprising: a) determining a first expression level of at least one gene from at least one gene set in a biological sample collected from the subject at a first time point, wherein the first time point is prior to the administration of at least one therapeutically effective amount of at least one SMARCA4-targeting compound; b) determining a second expression level of the least one gene in a biological sample collected from the subject at a second time point, wherein the second time point is after the administration of at least one therapeutically effective amount of at least one SMARCA4-targeting compound; c) comparing the second expression level of the at least one gene to the first expression level of the at least one gene; and d) administering to the subject at least one additional therapeutically effective amount of the at least one SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times less than the first expression level of the at least one gene, or else administering at least one alternative therapy to the subject. In some embodiments of the preceding method, the at least one gene is selected from the group consisting of the genes recited in Table 3. In some embodiments of the preceding method, the at least one gene set is selected from the gene sets recited in Table 4.

In some aspects, the present disclosure provides a method of treating a cancer in a subject, the method comprising: a) determining a first expression level of the genes from at least one gene set in a biological sample collected from the subject at a first time point, wherein the first time point is prior to the administration of at least one therapeutically effective amount of at least one SMARCA4-targeting compound; b) determining a second expression level of the genes from the at least one gene set in a biological sample collected from the subject at a second time point, wherein the second time point is after the administration of at least one therapeutically effective amount of at least one SMARCA4-targeting compound; c) determining whether the at least one gene set is downregulated in the biological sample collected from the subject at the second time point as compared to the biological sample collected from the subject at the first time point, based on the expression levels measured in steps (a) and (b); and d) administering to the subject at least one additional therapeutically effective amount of the at least one SMARCA4-targeting compound when the at least one gene set is downregulated in the biological sample collected from the subject at the second time point as compared to the biological sample collected from the subject at the first time point, or else administering at least one alternative therapy to the subject when the at least one gene set is not downregulated in the biological sample collected from the subject at the second time point as compared to the biological sample collected from the subject at the first time point.

In some aspects, the present disclosure provides a method of identifying at least one SMARCA4-targeting compound, the method comprising: a) treating at least one cell with at least one amount of at least one test compound, wherein the at least one cell exhibits aberrant SMARCA2 expression, activity or a combination thereof; b) determining the expression level of at least one gene from at least one gene set in the at least one cell; c) comparing the expression level of the at least one gene to at least one corresponding predetermined cutoff value; and d) identifying the at least one test compound as a SMARCA4-targeting compound when the expression level of the at least one gene is greater than the at least one corresponding predetermined cutoff value. In some embodiments of the preceding method, the at least one gene is selected from the group consisting of the genes recited in Table 1. In some embodiments of the preceding method, the at least one gene set is selected from the gene sets recited in Table 2.

In some aspects, the present disclosure provides a method of identifying at least one SMARCA4-targeting compound, the method comprising: a) treating at least one cell with at least one amount of at least one test compound, wherein the at least one cell exhibits aberrant SMARCA2 expression, activity or a combination thereof; b) determining the expression level of at least one gene from at least one gene set in the at least one cell; c) comparing the expression level of the at least one gene to at least one corresponding predetermined cutoff value; and d) identifying the at least one test compound as a SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times greater than the at least one corresponding predetermined cutoff value. In some embodiments of the preceding method, the at least one gene is selected from the group consisting of the genes recited in Table 1. In some embodiments of the preceding method, the at least one gene set is selected from the gene sets recited in Table 2.

In some aspects, the present disclosure provides a method of identifying at least one SMARCA4-targeting compound, the method comprising: a) treating at least one cell with at least one amount of at least one test compound, wherein the at least one cell exhibits aberrant SMARCA2 expression, activity or a combination thereof; b) determining the expression level of the genes from at least one gene set in the at least one treated cell; c) determining whether the at least one gene set is upregulated in the biological sample as compared to a reference sample, based on the expression levels measured in step (b); and d) identifying the at least one test compound as a SMARCA4-targeting compound when the at least one gene set is upregulated in the at least one treated cell as compared to the reference sample.

In some aspects, the present disclosure provides a method of identifying at least one SMARCA4-targeting compound, the method comprising: a) treating at least one cell with at least one amount of at least one test compound, wherein the at least one cell exhibits aberrant SMARCA2 expression, activity or a combination thereof; b) determining the expression level of at least one gene from at least one gene set in the at least treated one cell; c) comparing the expression level of the at least one gene to at least one corresponding predetermined cutoff value; and d) identifying the at least one test compound as a SMARCA4-targeting compound when the expression level of the at least one gene is less than the at least one corresponding predetermined cutoff value. In some embodiments of the preceding method, the at least one gene is selected from the group consisting of the genes recited in Table 3. In some embodiments of the preceding method, the at least one gene set is selected from the gene sets recited in Table 4.

In some aspects, the present disclosure provides a method of identifying at least one SMARCA4-targeting compound, the method comprising: a) treating at least one cell with at least one amount of at least one test compound, wherein the at least one cell exhibits aberrant SMARCA2 expression, activity or a combination thereof; b) determining the expression level of at least one gene from at least one gene set in the at least treated one cell; c) comparing the expression level of the at least one gene to at least one corresponding predetermined cutoff value; and d) identifying the at least one test compound as a SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times less than the at least one corresponding predetermined cutoff value. In some embodiments of the preceding method, the at least one gene is selected from the group consisting of the genes recited in Table 3. In some embodiments of the preceding method, the at least one gene set is selected from the gene sets recited in Table 4.

In some aspects, the present disclosure provides a method of identifying at least one SMARCA4-targeting compound, the method comprising: a) treating at least one cell with at least one amount of at least one test compound, wherein the at least one cell exhibits aberrant SMARCA2 expression, activity or a combination thereof; b) determining the expression level of the genes from at least one gene set in the at least one treated cell; c) determining whether the at least one gene set is downregulated in the biological sample as compared to a reference sample, based on the expression levels measured in step (b); and d) identifying the at least one test compound as a SMARCA4-targeting compound when the at least one gene set is downregulated in the at least one treated cell as compared to the reference sample.

In some embodiments of the preceding methods, the at least one cell is a plurality of cells.

In some aspects, the present disclosure provides a method of identifying at least one SMARCA4-targeting compound, the method comprising: a) determining a first expression level of at least one gene from at least one gene set in a plurality of cells at a first time point, wherein the plurality of cells exhibits aberrant SMARCA2 expression, activity or a combination thereof; b) treating the plurality of cells with at least one amount of at least one test compound; c) determining a second expression level of the least one gene in the plurality of cells at a second time point; d) comparing the second expression level of the at least one gene to the first expression level of the at least one gene; and e) identifying the at least one test compound as a SMARCA4-targeting compound when the second expression level of the at least one gene is greater than the first expression level of the at least one gene. In some embodiments of the preceding method, the at least one gene is selected from the group consisting of the genes recited in Table 1. In some embodiments of the preceding method, the at least one gene set is selected from the gene sets recited in Table 2.

In some aspects, the present disclosure provides a method of identifying at least one SMARCA4-targeting compound, the method comprising: a) determining a first expression level of at least one gene from at least one gene set in a plurality of cells at a first time point, wherein the plurality of cells exhibits aberrant SMARCA2 expression, activity or a combination thereof; b) treating the plurality of cells with at least one amount of at least one test compound; c) determining a second expression level of the least one gene in the plurality of cells at a second time point; d) comparing the second expression level of the at least one gene to the first expression level of the at least one gene; and e) identifying the at least one test compound as a SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times greater than the first expression level of the at least one gene. In some embodiments of the preceding method, the at least one gene is selected from the group consisting of the genes recited in Table 1. In some embodiments of the preceding method, the at least one gene set is selected from the gene sets recited in Table 2.

In some aspects, the present disclosure provides a method of identifying at least one SMARCA4-targeting compound, the method comprising: a) determining a first expression level of the genes from at least one gene set in a plurality of cells at a first time point, wherein the plurality of cells exhibits aberrant SMARCA2 expression, activity or a combination thereof; b) treating the plurality of cells with at least one amount of at least one test compound; c) determining a second expression level of the genes from the at least one gene set in the plurality of treated cells at a second time point; d) determining whether the at least one gene set is upregulated at the second time point as compared to the first time point; and e) identifying the at least one test compound as a SMARCA4-targeting compound when the at least one gene set is upregulated at the second time point as compared to the first time point.

In some aspects, the present disclosure provides a method of identifying at least one SMARCA4-targeting compound, the method comprising: a) determining a first expression level of at least one gene from at least one gene set in a plurality of cells at a first time point, wherein the plurality of cells exhibits aberrant SMARCA2 expression, activity or a combination thereof; b) treating the plurality of cells with at least one amount of at least one test compound; c) determining a second expression level of the least one gene in the plurality of treated cells at a second time point; d) comparing the second expression level of the at least one gene to the first expression level of the at least one gene; and e) identifying the at least one test compound as a SMARCA4-targeting compound when the second expression level of the at least one gene is less than the first expression level of the at least one gene. In some embodiments of the preceding method, the at least one gene is selected from the group consisting of the genes recited in Table 3. In some embodiments of the preceding method, the at least one gene set is selected from the gene sets recited in Table 4.

In some aspects, the present disclosure provides a method of identifying at least one SMARCA4-targeting compound, the method comprising: a) determining a first expression level of at least one gene from at least one gene set in a plurality of cells at a first time point, wherein the plurality of cells exhibits aberrant SMARCA2 expression, activity or a combination thereof; b) treating the plurality of cells with at least one amount of at least one test compound; c) determining a second expression level of the least one gene in the plurality of treated cells at a second time point; d) comparing the second expression level of the at least one gene to the first expression level of the at least one gene; and e) identifying the at least one test compound as a SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, or at least about 15 times, or at least about 20 times, or at least about 25 times, or at least about 30 times, or at least about 35 times, or at least about 40 times, or at least about 45 times, or at least about 50 times, or at least about 55 times, or at least about 60 times, or at least about 65 times, or at least about 70 times, or at least about 75 times, or at least about 80 times, or at least about 85 times, or at least about 90 times, or at least about 95 times, or at least about 100 times less than the first expression level of the at least one gene. In some embodiments of the preceding method, the at least one gene is selected from the group consisting of the genes recited in Table 3. In some embodiments of the preceding method, the at least one gene set is selected from the gene sets recited in Table 4.

In some aspects, the present disclosure provides a method of identifying at least one SMARCA4-targeting compound, the method comprising: a) determining a first expression level of the genes from at least one gene set in a plurality of cells at a first time point, wherein the plurality of cells exhibits aberrant SMARCA2 expression, activity or a combination thereof; b) treating the plurality of cells with at least one amount of at least one test compound; c) determining a second expression level of the genes from the at least one gene set in the plurality of treated cells at a second time point; d) determining whether the at least one gene set is downregulated at the second time point as compared to the first time point; and e) identifying the at least one test compound as a SMARCA4-targeting compound when the at least one gene set is downregulated at the second time point as compared to the first time point.

In some embodiments of the preceding methods, the at least one cell is a cancer cell.

In some aspects, the present disclosure provides a method of modulating an epithelial/mesenchymal state in at least one cell comprising contacting the at least one cell with an effective amount of at least one SMARCA4-targeting compound. In some embodiments of the preceding method, the SMARCA4-targeting compound is a SMARCA4 inhibitor. In some embodiments of the preceding method, the at least one SMARCA4-targeting compound also targets or inhibits at least one other gene, including, but not limited to, SMARCA2.

In some aspects of the preceding method, the at least one cell can exhibit aberrant SMARCA2 expression, activity or a combination thereof. In some aspects of the preceding method, the at least one cell can exhibit aberrant SMARCA4 expression, activity or a combination thereof.

In some aspects of the preceding method, modulating an epithelial/mesenchymal state in the at least one cell can comprise altering the expression level of at least one gene and/or protein associated with an epithelial state. In some embodiments, the at least one gene and/or protein associated with an epithelial state is E-cadherin, FOXA1 or CLDN1.

In some aspects of the preceding method, modulating an epithelial/mesenchymal state in the at least one cell can comprise altering the expression level of at least one gene and/or protein associated with a mesenchymal state. In some embodiments, the at least one gene and/or protein associated with a mesenchymal state is N-cadherin, vimentin, SNAI1 or ZEB1.

In some embodiments of the methods of the present disclosure, the gene set “HALLMARK_TGF_BETA_SIGNALING” can comprise, consist of, or essentially consist of the genes recited in Table 5.

TABLE 5

| | | |
|---|---|---|
| ACVR1 | MAP3K7 | TRIM33 |
| APC | NCOR2 | UBE2D3 |
| ARID4B | NOG | WWTR1 |
| BCAR3 | PMEPA1 | XIAP |
| BMP2 | PPM1A | |
| BMPR1A | PPP1CA | |
| BMPR2 | PPP1R15A | |
| CDH1 | RAB31 | |
| CDK9 | RHOA | |
| CDKN1C | SERPINE1 | |
| CTNNB1 | SKI | |
| ENG | SKIL | |
| FKBP1A | SLC20A1 | |
| FNTA | SMAD1 | |
| FURIN | SMAD3 | |
| HDAC1 | SMAD6 | |
| HIPK2 | SMAD7 | |
| ID1 | SMURF1 | |
| ID2 | SMURF2 | |
| ID3 | SPTBN1 | |
| IFNGR2 | TGFB1 | |
| JUNB | TGFBR1 | |
| KLF10 | TGIF1 | |
| LEFTY2 | THBS1 | |
| LTBP2 | TJP1 | |

In some embodiments of the methods of the present disclosure, the gene set “HALLMARK_E2F_TARGETS” can comprise, consist of, or essentially consist of the genes recited in Table 6.

TABLE 6

| | | | | | | | |
|---|---|---|---|---|---|---|---|
| AK2 | CDKN1B | DUT | KIF22 | MYC | POLA2 | RFC1 | SYNCRIP |
| ANP32E | CDKN2A | E2F8 | KIF2C | NAA38 | POLD1 | RFC2 | TACC3 |
| ASF1A | CDKN2C | EED | KIF4A | NAPIL1 | POLD2 | RFC3 | TBRG4 |
| ASF1B | CDKN3 | EIF2S1 | KPNA2 | NASP | POLD3 | RNASEH2A | TCF19 |
| ATAD2 | CENPE | ESPL1 | LBR | NBN | POLE | RPA1 | TFRC |
| AURKA | CENPM | EXOSC8 | LIG1 | NCAPD2 | POLE4 | RPA2 | TIMELESS |
| AURKB | CHEK1 | EZH2 | LMNB1 | NME1 | POP7 | RPA3 | TIP1N |
| BARD1 | CHEK2 | GINS1 | LUC7L3 | NOLC1 | PPM1D | RQCD1 | TK1 |
| BIRC5 | CIT | GINS3 | LYAR | NOP56 | PPP1R8 | RRM2 | TMPO |
| BRCA1 | CKS1B | GINS4 | MAD2L1 | NUDT21 | PRDX4 | SHMT1 | TOP2A |
| BRCA2 | CKS2 | GSPT1 | MCM2 | NUP107 | PRIM2 | SLBP | TP53 |
| BRMS1L | CSE1L | H2AFX | MCM3 | NUP153 | PRKDC | SMC1A | TRA2B |
| BUB1B | CTCF | H2AFZ | MCM4 | NUP205 | PRPS1 | SMC3 | TR1P13 |
| CBX5 | CTPS | HELLS | MCM5 | ORC2 | PSIP1 | SMC4 | TUBB |
| CCNB2 | DCK | HMGA1 | MCM6 | ORC6 | PSMC3IP | SMC6 | TUBG1 |
| CCNE1 | DCLRE1B | HMGB2 | MCM7 | PA2G4 | PTTG1 | SNRPB | UBE2S |
| CCP110 | DCTPP1 | HMGB3 | MELK | PAICS | RACGAP1 | SPAG5 | UBE2T |
| CDC20 | DDX39A | HMMR | MKI67 | PAN2 | RAD1 | SPC24 | UBR7 |
| CDC25A | DEK | HN1 | MLH1 | PCNA | RAD21 | SPC25 | UNG |
| CDC25B | DEPDC1 | HNRNPD | MMS22L | PDS5B | RAD50 | SRSF1 | USP1 |
| CDCA3 | DIAPH3 | HUS1 | MRE11A | PHP5A | RAD51AP1 | SRSF2 | WDR90 |
| CDCA8 | DLGAP5 | ILF3 | MSH2 | PLK1 | RAD51C | SSRP1 | WEE1 |
| CDK1 | DNMT1 | ING3 | MTHFD2 | PLK4 | RAN | STAG1 | XPO1 |
| CDK4 | DONSON | IPO7 | MXD3 | PMS2 | RANBP1 | STMN1 | XRCC6 |
| CDKN1A | DSCC1 | K1F18B | MYBL2 | PNN | RBBP7 | SUV39H1 | ZW10 |

In some embodiments of the methods of the present disclosure, the gene set “HALLMARK_G2M_CHECKPOINT” can comprise, consist of, or essentially consist of the genes recited in Table 7.

TABLE 7

| | | | | | | | |
|---|---|---|---|---|---|---|---|
| ABL1 | CDC45 | DR1 | HMGB3 | MAD2L1 | ODC1 | RASAL2 | STMN1 |
| AMD1 | CDC6 | DTYMK | HMGN2 | MAPK14 | ODF2 | RBL1 | SUV39H1 |
| ARID4A | CDC7 | E2F1 | HMMR | MARCKS | ORC5 | RBM14 | SYNCRIP |
| ATF5 | CDK1 | E2F2 | HN1 | MCM2 | ORC6 | RPA2 | TACC3 |
| ATRX | CDK4 | E2F3 | HNRNPD | MCM3 | PAFAH1B1 | RPS6KA5 | TFDP1 |
| AURKA | CDKN1B | E2F4 | HNRNPU | MCM5 | PAPD7 | SAP30 | TGFB1 |
| AURKB | CDKN2C | EFNA5 | HOXC10 | MCM6 | PBK | SETD8 | TLE3 |
| BARD1 | CDKN3 | EGF | HSPA8 | MEIS1 | PDS5B | SFPQ | TMPO |
| BCL3 | CENPA | ESPL1 | HUS1 | MEIS2 | PLK1 | SLC12A2 | TNPO2 |
| BIRC5 | CENPE | EWSR1 | ILF3 | MKI67 | PLK4 | SLC38A1 | TOP1 |
| BRCA2 | CENPF | EXO1 | INCENP | MNAT1 | PML | SLC7A1 | TOP2A |
| BUB1 | CHAF1A | EZH2 | KATNA1 | MT2A | POLA2 | SLC7A5 | TPX2 |
| BUB3 | CHEK1 | FANCC | KIF11 | MTF2 | POLE | SMAD3 | TRA2B |
| CASC5 | CHMP1A | FBXO5 | KIF15 | MYBL2 | POLQ | SMARCC1 | TRAIP |
| CASP8AP2 | CKS1B | FOXN3 | KIF20B | MYC | PRC1 | SMC1A | TROAP |
| CBX1 | CKS2 | G3BP1 | KIF22 | NASP | PRIM2 | SMC2 | TTK |
| CCNA2 | CTCF | GINS2 | KIF23 | NCL | PRMT5 | SMC4 | UBE2C |
| CCNB2 | CUL1 | GSPT1 | KIF2C | NDC80 | PRPF4B | SNRPD1 | UBE2S |
| CCND1 | CUL3 | H2AFV | KIF4A | NEK2 | PTTG1 | SQLE | UCK2 |
| CCNF | CUL4A | H2AFX | KIF5B | NOLC1 | PTTG3P | SRSF1 | UPF1 |
| CCNT1 | CUL5 | H2AFZ | KPNA2 | NOTCH2 | PURA | SRSF10 | WHSC1 |
| CDC20 | DBF4 | HIF1A | KPNB1 | NUMA1 | RACGAP1 | SRSF2 | WRN |
| CDC25A | DDX39A | HIRA | LBR | NUP50 | RAD21 | SS18 | XPO1 |
| CDC25B | DKC1 | HIST1H2BK | LIG3 | NUP98 | RAD23B | STAG1 | YTHDC1 |
| CDC27 | DMD | HMGA1 | LMNB1 | NUSAP1 | RAD54L | STIL | ZAK |

In some embodiments of the methods of the present disclosure, the gene set “HALLMARK_MYC_TARGETS_V1” can comprise, consist of, or essentially consist of the genes recited in Table 8.

TABLE 8

| | | | | | | | |
|---|---|---|---|---|---|---|---|
| ABCE1 | CTPS | G3BP1 | KARS | NPM1 | PSMB3 | RPS6 | SSBP1 |
| ACP1 | CUL1 | GLO1 | KPNA2 | ODC1 | PSMC4 | RRM1 | STARD7 |
| AIMP2 | CYC1 | GNB2L1 | KPNB1 | ORC2 | PSMC6 | RRP9 | SYNCRIP |
| AP3S1 | DDX18 | GNL3 | LDHA | PA2G4 | PSMD1 | RSL1D1 | TARDBP |
| APEX1 | DDX21 | GOT2 | LSM2 | PABPC1 | PSMD14 | RUVBL2 | TCP1 |
| BUB3 | DEK | GSPT1 | LSM7 | PABPC4 | PSMD3 | SERBP1 | TFDP1 |
| C1QBP | DHX15 | H2AFZ | MAD2L1 | PCBP1 | PSMD7 | SET | TOMM70A |
| CAD | DUT | HDAC2 | MCM2 | PCNA | PSMD8 | SF3A1 | TRA2B |
| CANX | EEF1B2 | HDDC2 | MCM4 | PGK1 | PTGES3 | SF3B3 | TRIM28 |
| CBX3 | EIF1AX | HDGF | MCM5 | PHB | PWP1 | SLC25A3 | TUFM |
| CCNA2 | EIF2S1 | HNRNPA1 | MCM6 | PHB2 | RAD23B | SMARCC1 | TXNL4A |
| CCT2 | EIF2S2 | HNRNPA2B1 | MCM7 | POLD2 | RAN | SNRPA | TYMS |
| CCT3 | EIF3B | HNRNPA3 | MRPL23 | POLE3 | RANBP1 | SNRPA1 | U2AF1 |
| CCT4 | EIF3D | HNRNPC | MRPL9 | PPIA | RFC4 | SNRPB2 | UBA2 |
| CCT5 | EIF3J | HNRNPD | MRPS18B | PPM1G | RNPS1 | SNRPD1 | UBE2E1 |
| CCT7 | EIF4A1 | HNRNPR | MYC | PRDX3 | RPL14 | SNRPD2 | UBE2L3 |
| CDC20 | EIF4E | HNRNPU | NAP1L1 | PRDX4 | RPL18 | SNRPD3 | USP1 |
| CDC45 | EIF4G2 | HPRT1 | NCBP1 | PRPF31 | RPL22 | SNRPG | VBP1 |
| CDK2 | EIF4H | HSP90AB1 | NCBP2 | PRPS2 | RPL34 | SRM | VDAC1 |
| CDK4 | EPRS | HSPD1 | NDUFAB1 | PSMA1 | RPL6 | SRPK1 | VDAC3 |
| CLNS1A | ERH | HSPE1 | NHP2 | PSMA2 | RPLP0 | SRSF1 | XPO1 |
| CNBP | ETF1 | IARS | NME1 | PSMA4 | RPS10 | SRSF2 | XPOT |
| COPS5 | EXOSC7 | IFRD1 | NOLC1 | PSMA6 | RPS2 | SRSF3 | XRCC6 |
| COX5A | FAM120A | ILF2 | NOP16 | PSMA7 | RPS3 | SRSF7 | YWHAE |
| CSTF2 | FBL | IMPDH2 | NOP56 | PSMB2 | RPS5 | SSB | YWHAQ |

In some embodiments of the methods of the present disclosure, the gene set “HALLMARK_MTORC1_SIGNALING” can comprise, consist of, or essentially consist of the genes recited in Table 9.

TABLE 9

| | | | | | | | |
|---|---|---|---|---|---|---|---|
| ABCF2 | CCT6A | ELOVL6 | HMBS | MCM4 | PNP | RRM2 | STARD4 |
| ACACA | CD9 | ENO1 | HMGCR | ME1 | POLR3G | RRP9 | STC1 |
| ACLY | CDC25A | EPRS | HMGCS1 | MLLT11 | PPA1 | SC5DL | STIP1 |
| ACSL3 | CDKN1A | ERO1L | HPRT1 | MTHFD2 | PPIA | SCD | SYTL2 |
| ACTR2 | CFP | ETF1 | HSP90B1 | MTHFD2L | PPP1R15A | SDF2L1 | TBK1 |
| ACTR3 | COPS5 | FADS1 | HSPA4 | NAMPT | PRDX1 | SEC11A | TCEA1 |
| ADD3 | CORO1A | FADS2 | HSPA5 | NFIL3 | PSAT1 | SERP1 | TES |
| ADIPOR2 | CTH | FAM129A | HSPA9 | NFKBIB | PSMA3 | SERPINH1 | TFRC |
| AK4 | CTSC | FDXR | HSPD1 | NFYC | PSMA4 | SHMT2 | TM7SF2 |
| ALDOA | CXCR4 | FGL2 | HSPE1 | NMT1 | PSMB5 | SKAP2 | TMEM97 |
| ARPC5L | CYB5B | FKBP2 | IDH1 | NUFIP1 | PSMC2 | SLA | TOMM40 |
| ASNS | CYP51A1 | G6PD | IDI1 | NUP205 | PSMC4 | SLC1A4 | TPI1 |
| ATP2A2 | DAPP1 | GAPDH | IFI30 | NUPR1 | PSMC6 | SLC1A5 | TRIB3 |
| ATP5G1 | DD1T3 | GBE1 | IFRD1 | P4HA1 | PSMD12 | SLC2A1 | TUBA4A |
| ATP6V1D | DD1T4 | GCLC | IGFBP5 | PDAP1 | PSMD13 | SLC2A3 | TUBG1 |
| AURKA | DDX39A | GGA2 | IMMT | PDK1 | PSMD14 | SLC37A4 | TXNRD1 |
| BCAT1 | DHCR24 | GLA | INSIG1 | PFKL | PSME3 | SLC6A6 | UBE2D3 |
| BHLHE40 | DHCR7 | GLRX | ITGB2 | PGK1 | PSMG1 | SLC7A11 | UCHL5 |
| BTG2 | DHFR | GMPS | LDHA | PGM1 | PSPH | SLC7A5 | UFM1 |
| BUB1 | EBP | GOT1 | LDLR | PHGDH | QDPR | SLC9A3R1 | UNG |
| CACYBP | EDEM1 | GPI | LGMN | PIK3R3 | RAB1A | SORD | USO1 |
| CALR | EEF1E1 | GSK3B | LTA4H | PITPNB | RDH11 | SQLE | VLDLR |
| CANX | EGLN3 | GSR | M6PR | PLK1 | RIT1 | SQSTM1 | WARS |
| CCNF | EIF2S2 | GTF2H1 | MAP2K3 | PLOD2 | RPA1 | SRD5A1 | XBP1 |
| CCNG1 | ELOVLS | HK2 | MCM2 | PNO1 | RPN1 | SSR1 | YKT6 |

In some embodiments of the methods of the present disclosure, the gene set “HALLMARK_INTERFERON_ALPHA_RESPONSE” can comprise, consist of, or essentially consist of the genes recited in Table 10.

TABLE 10

| | | | |
|---|---|---|---|
| ADAR | GMPR | LGALS3BP | RSAD2 |
| B2M | HERC6 | LPAR6 | RTP4 |
| BATF2 | HLA-C | LY6E | SAMD9 |
| BST2 | IFI27 | MOV10 | SAMD9L |
| C1S | IFI30 | MX1 | SELL |
| CASP1 | IFI35 | NCOA7 | SLC25A28 |
| CASP8 | IFI44 | NMI | SP110 |
| CCRL2 | IFI44L | NUB1 | STAT2 |
| CD47 | IFIH1 | OAS1 | TAP1 |
| CD74 | IFIT2 | OASL | TDRD7 |
| CMPK2 | IFIT3 | OGFR | TMEM140 |
| CNP | IFITM1 | PARP12 | TRAFD1 |
| CSF1 | IFITM2 | PARP14 | TRIM14 |
| CXCL10 | IFITM3 | PARP9 | TRIM21 |
| CXCL11 | IL15 | PLSCR1 | TRIM25 |
| DDX60 | IL4R | PNPT1 | TRIM26 |
| DHX58 | IL7 | PRIC285 | TRIMS |
| EIF2AK2 | IRF1 | PROCR | TXNIP |
| ELF1 | IRF2 | PSMA3 | UBA7 |
| EPSTI1 | IRF7 | PSMB8 | UBE2L6 |
| FAM125A | IRF9 | PSMB9 | USP18 |
| FAM46A | ISG15 | PSME1 | WARS |
| FTSJD2 | ISG20 | PSME2 | |
| GBP2 | LAMP3 | RIPK2 | |
| GBP4 | LAP3 | RNF31 | |

In some embodiments of the methods of the present disclosure, the gene set “HALLMARK_MYC_TARGETS_V2” can comprise, consist of, or essentially consist of the genes recited in Table 11.

TABLE 11

| | | |
|---|---|---|
| AIMP2 | NIP7 | TBRG4 |
| BYSL | NOC4L | TCOF1 |
| CBX3 | NOLC1 | TFB2M |
| CDK4 | NOP16 | TMEM97 |
| DCTPP1 | NOP2 | UNG |
| DDX18 | NOP56 | UTP20 |
| DUSP2 | NPM1 | WDR43 |
| EXOSC5 | PA2G4 | WDR74 |
| FARSA | PES1 | |
| GNL3 | PHB | |
| GRWD1 | PLKI | |
| HK2 | PLK4 | |
| HSPD1 | PPAN | |
| HSPE1 | PPRCI | |
| IMP4 | PRMT3 | |
| IPO4 | PUS1 | |
| LAS1L | RABEPK | |
| MAP3K6 | RCL1 | |
| MCM4 | RRP12 | |
| MCM5 | RRP9 | |
| MPHOSPH10 | SLC19A1 | |
| MRTO4 | SLC29A2 | |
| MYBBP1A | SORD | |
| MYC | SRM | |
| NDUFAF4 | SUPV3L1 | |

In some embodiments of the methods of the present disclosure, the gene set “HALLMARK_INTERFERON_GAMMA_RESPONSE” can comprise, consist of, or essentially consist of the genes recited in Table 12.

TABLE 12

| | | | | | | | |
|---|---|---|---|---|---|---|---|
| ADAR | CD74 | GCH1 | IL10RA | LY6E | PDE4B | RIPK2 | TAP1 |
| APOL6 | CD86 | GPR18 | IL15 | LYSMD2 | PELI1 | RNF213 | TAPBP |
| ARID5B | CDKN1A | GZMA | IL15RA | 43891 | PFKP | RNF31 | TDRD7 |
| ARL4A | CFB | HERC6 | IL18BP | METTL7B | PIM1 | RSAD2 | TNFAIP2 |
| AUTS2 | CFH | HIF1A | IL2RB | MT2A | PLA2G4A | RTP4 | TNFAIP3 |
| B2M | CIITA | HLA-A | IL4R | MTHFD2 | PLSCR1 | SAMD9L | TNFAIP6 |
| BANK1 | CMKLR1 | HLA-B | IL6 | MVP | PML | SAMHD1 | TNFSF10 |
| BATF2 | CMPK2 | HLA- | IL7 | MX1 | PNP | SECTM1 | TOR1B |
| | | DMA | | | | | |
| BPGM | CSF2RB | HLA- | IRF1 | MX2 | PNPT1 | SELP | TRAFD1 |
| | | DOA1 | | | | | |
| BST2 | CXCL10 | HLA- | IRF2 | MYD88 | PRIC285 | SERPING1 | TRIM14 |
| | | DRB1 | | | | | |
| BTG1 | CXCL11 | HLA-G | IRF4 | NAMPT | PSMA2 | SLAMF7 | TRIM21 |
| C1R | CXCL9 | ICAM1 | IRF5 | NCOA3 | PSMA3 | SLC25A28 | TRIM25 |
| C1S | DDX58 | IDO1 | IRF7 | NFKB1 | PSMB10 | SOCS1 | TRIM26 |
| CASP1 | DDX60 | IFI27 | IRF8 | NFKBIA | PSMB2 | SOCS3 | TXNIP |
| CASP3 | DHX58 | IFI30 | IRF9 | NLRC5 | PSMB8 | SOD2 | UBE2L6 |
| CASP4 | EIF2AK2 | IFI35 | ISG15 | NMI | PSMB9 | SP110 | UPP1 |
| CASP7 | EIF4E3 | IFI44 | ISG20 | NOD1 | PSME1 | SPPL2A | USP18 |
| CASP8 | EPSTI1 | IFI44L | ISOC1 | NUP93 | PSME2 | SRI | VAMP5 |
| CCL2 | FAS | IFIH1 | ITGB7 | OAS2 | PTGS2 | SSPN | VAMP8 |
| CCL5 | FCGR1A | IFIT1 | JAK2 | OAS3 | PTPN1 | ST3GAL5 | VCAM1 |
| CCL7 | FGL2 | IFIT2 | KLRK1 | OASL | PTPN2 | ST8SIA4 | WARS |
| CD274 | FPR1 | IFIT3 | LAP3 | OGFR | PTPN6 | STAT1 | XAF1 |
| CD38 | FTSJD2 | IFITM2 | LATS2 | P2RY14 | RAPGEF6 | STAT2 | XCL1 |
| CD40 | GBP4 | IFITM3 | LCP2 | PARP12 | RBCK1 | STAT3 | ZBP1 |
| CD69 | GBP6 | IFNAR2 | LGALS3BP | PARP14 | RIPK1 | STAT4 | ZNFX1 |

In some embodiments of the methods of the present disclosure, an alternative therapy can comprise a therapy that does not include the administration of a SMARCA4-targeting compound. Alternative therapies can include, but are not limited to, radiation therapy, surgery, chemotherapy, immunotherapy, hormone therapy, cryoablation, radiofrequency ablation, targeted drug therapy or any combination thereof.

In some aspects of the methods of the present disclosure, determining the expression level of at least one gene from at least one gene set can comprise determining the expression of at least one, or at least two, or at least three, or at least four, or at least five, or at least six, or at least seven, or at least eight, or at least nine, or at least ten, or at least 15, or at least 20, or at least 25, or at least 30, or at least 35, or at least 40, or at least 45, or at least 50, or at least 55, or at least 60, or at least 65, or at least 70, or at least 75, or at least 80, or at least 85, or at least 90, or at least 95, or at least 100, or at least 105, or at least 110, or at least or at least 115, or at least 120, or at least 125, or at least 130, or at least 135, or at least 140, or at least 145, or at least 150, or at least 155, or at least 160, or at least 165, or at least 170, or at least 175, or at least 180, or at least 185, or at least 190, or at least 195, or at least 200 genes from at least one gene set. In some aspects of the methods of the present disclosure, determining the expression level of at least one gene from at least one gene set can comprise determining the expression level of all of the genes in the gene set.

In some aspects of the methods of the present disclosure, determining the expression level of at least one gene from at least one gene set can comprise determining the expression of at least one gene from at least one, or at least two, or at least three, or at least four, or at least five, or at least six, or at least seven, or at least eight, or at least nine, or at least ten gene sets.

In some embodiments of the methods of the present disclosure, determining the expression level of at least one gene comprises determining the mRNA expression level of the at least one gene. In some embodiments of the methods of the present disclosure, determining the expression level of at least one gene comprises determining the protein expression level of the at least one gene. In some embodiments of the methods of the present disclosure, determining the expression level of at least one gene comprises determining the mRNA expression level and the protein expression level of the at least one gene.

As would be appreciated by those of ordinary skill in the art, determining the expression level of a gene or of a plurality of genes can comprise PCR, targeted sequencing, high-throughput sequencing, next generation sequencing, Northern Blot, reverse transcription PCR (RT-PCR), real-time PCR (qPCR), quantitative PCR, qRT-PCR, flow cytometry, mass spectrometry, microarray analysis, digital droplet PCR, Western Blot or any combination thereof.

As would be appreciated by the those of ordinary skill in the art, determining whether the gene set is upregulated or downregulated in the biological sample as compared to a reference sample can comprise performing gene set enrichment analysis (GSEA) (e.g., see Subrmanian, Tamayo, et al. PNAS, 2005, 102, pgs 15545-15550; Liberzon, Arthur, et al. Bioinformatics, 2011, 27(12), pgs 1739-1740; Liberzon, Arthur, et al. Cell Systems, 2015, 1(6), pgs 417-425). Those of ordinary skill in the art will be aware of methods and/or tools for performing GSEA, including, but not limited to, Nucleic Acid SeQuence Analysis Resource (NASQAR), MSigDB, WebGestalt, Enrichr, GeneSCF, DAVID, Metascape, AmiGO 2, genomic region enrichment of annotations tool (GREAT), Functional Enrichment Analysis (FunRich), InterMine, ToppGene, quantitative set analysis for gene expression (QuSage), Blast2GO and g:Profiler.

Those of ordinary skill in the art will be aware of the individual genes that are part of the gene sets enumerated herein. In a non-limiting example, those of ordinary skill in the art will be aware that the individual genes that are part of a particular gene set enumerated herein can be determined by consulting the relevant database for that particular gene set. Those of ordinary skill in the art will be aware that such databases include, but are not limited to, MSigDB (e.g., see Liberzon, Arthur, et al. Bioinformatics, 2011, 27(12), pgs 1739-1740; Liberzon, Arthur, et al. Cell Systems, 2015, 1(6), pgs 417-425).

In some embodiments of the methods of the present disclosure, a gene set is said to be upregulated or downregulated if the familywise-error rate (FWER) p-value is less than 0.05. In some embodiments of the methods of the present disclosure, a gene set is said to be upregulated or downregulated if the FWER p-value is less than about 0.1, or less than about 0.05, or less than about 0.01, or less than about 0.005, or less than about 0.001, or less than about 0.0005 or less than about 0.0001.

In some embodiments of the methods of the present disclosure, a gene set is said to be upregulated or downregulated if the false discovery rate-adjusted p-values (q-value) is less than 0.05. In some embodiments of the methods of the present disclosure, a gene set is said to be upregulated or downregulated if the false discovery rate-adjusted p-values (q-value) is less than about 0.1, or less than about 0.05, or less than about 0.01, or less than about 0.005, or less than about 0.001, or less than about 0.0005 or less than about 0.0001.

In some aspects, a predetermined cutoff value can be the expression level of at least one gene from at least one gene set in a reference sample. In some aspects, a predetermined cutoff value can be the average (mean) expression level of at least one gene from at least one gene set in a plurality reference samples.

In some embodiments of the methods of the present disclosure, a reference sample is a sample collected from a subject who was previously identified as being responsive to therapy comprising the administration of a SMARCA4-targeting compound. In some embodiments of the methods of the present disclosure, a reference sample is a sample collected from a subject who was previously identified as being non-responsive to therapy comprising the administration of a SMARCA4-targeting compound. In some embodiments of the methods of the present disclosure, a reference sample is a sample from a cell contacted with a compound that is known to target SMARCA4. In some embodiments, a reference sample can be comprise a plurality of reference samples from a plurality of subjects.

In some aspects of the disclosure, a SMARCA4-targeting compound is any SMARCA4-targeting compound known and appreciated in the art. In some embodiments, the SMARCA4-targeting compound is a compound recited in WO/2020/023657, the entire contents of which are incorporated herein by reference.

In some aspects of the disclosure, a SMARCA4-targeting compound can be a SMARCA4 inhibitor. As used herein, a SMARCA4 inhibitor can also be referred to as a SMARCA4 antagonist. In some aspects of the disclosure, a SMARCA4-targeting compound can be a SMARCA4 degrader.

In certain aspects of the disclosure, the inhibitor targets the helicase domain of SMARCA4. In some embodiments, the inhibitor targets the ATP domain of SMARCA4. In some embodiments, the inhibitor does not target the bromodomain of SMARCA4 In some embodiments, the inhibitor targets the bromodomain of SMARCA4.

In some aspects, a SMARCA4 inhibitor inhibits SMARCA4 helicase activity. In some embodiments, a SMARCA4 inhibitor inhibits SMARCA4 helicase activity by at least 10%. In some embodiments, a SMARCA4 inhibitor inhibits SMARCA4 helicase activity by at least 20%. In some embodiments, a SMARCA4 inhibitor inhibits SMARCA4 helicase activity by at least 30% In some embodiments, a SMARCA4 inhibitor inhibits SMARCA4 helicase activity by at least 40% In some embodiments, a SMARCA4 inhibitor inhibits SMARCA4 helicase activity by at least 50% In some embodiments, a SMARCA4 inhibitor inhibits SMARCA4 helicase activity by at least 60%. In some embodiments, a SMARCA4 inhibitor inhibits SMARCA4 helicase activity by at least 70% In some embodiments, a SMARCA4 inhibitor inhibits SMARCA4 helicase activity by at least 80%. In some embodiments, a SMARCA4 inhibitor inhibits SMARCA4 helicase activity by at least 90%. In some embodiments, a SMARCA4 inhibitor inhibits SMARCA4 helicase activity by at least 95%. In some embodiments, a SMARCA4 inhibitor inhibits SMARCA4 helicase activity by at least 98%. In some embodiments, a SMARCA4 inhibitor inhibits SMARCA4 helicase activity by or at least 99%. In some embodiments, a SMARCA4 inhibitor inhibits SMARCA4 helicase activity and abolishes SMARCA4 activity.

In some aspects, a SMARCA4 inhibitor inhibits SMARCA4 ATPase activity. In some embodiments, a SMARCA4 inhibitor inhibits SMARCA4 ATPase activity by at least 10%. In some embodiments, a SMARCA4 inhibitor inhibits SMARCA4 ATPase activity by at least 20%. In some embodiments, a SMARCA4 inhibitor inhibits SMARCA4 ATPase activity by at least 30%. In some embodiments, a SMARCA4 inhibitor inhibits SMARCA4 ATPase activity by at least 40%. In some embodiments, a SMARCA4 inhibitor inhibits SMARCA4 ATPase activity by at least 50%. In some embodiments, a SMARCA4 inhibitor inhibits SMARCA4 ATPase activity by at least 60%. In some embodiments, a SMARCA4 inhibitor inhibits SMARCA4 ATPase activity by at least 70%. In some embodiments, a SMARCA4 inhibitor inhibits SMARCA4 ATPase activity by at least 80%. In some embodiments, a SMARCA4 inhibitor inhibits SMARCA4 ATPase activity by at least 90%. In some embodiments, a SMARCA4 inhibitor inhibits SMARCA4 ATPase activity by at least 95%. In some embodiments, a SMARCA4 inhibitor inhibits SMARCA4 ATPase activity by at least 98% In some embodiments, a SMARCA4 inhibitor inhibits SMARCA4 ATPase activity by or at least 99%. In some embodiments, a SMARCA4 inhibitor inhibits SMARCA4 ATPase activity and abolishes SMARCA4 activity.

In certain aspects of the disclosure, the SMARCA4 inhibitor inhibits SMARCA4 activity. Inhibition of SMARCA4 activity can be detected using any suitable method. The inhibition can be measured, for example, either in terms of rate of SMARCA4 activity or as product of SMARCA4 activity.

The inhibition is a measurable inhibition compared to a suitable control. In some embodiments, inhibition is at least 10 percent inhibition compared to a suitable control. That is, the rate of enzymatic activity or the amount of product with the inhibitor is less than or equal to 90 percent of the corresponding rate or amount made without the inhibitor. In some embodiments, inhibition is at least 20, 25, 30, 40, 50, 60, 70, 75, 80, 90, or 95 percent inhibition compared to a suitable control. In some embodiments, inhibition is at least 99 percent inhibition compared to a suitable control. That is, the rate of enzymatic activity or the amount of product with the inhibitor is less than or equal to 1 percent of the corresponding rate or amount made without the inhibitor.

In some aspects, a SMARCA4-targeting compound may also target another gene. In some embodiments, the SMARCA4-targeting compound may also be a SMARCA2-targeting compound (e.g., a SMARCA2 inhibitor, also referred to as a SMARCA2 antagonist).

In some embodiments of the methods of the present disclosure, a cancer exhibits aberrant SMARCA2 expression, activity, function or a combination thereof.

In some embodiments, aberrant SMARCA2 expression comprises decreased SMARCA2 expression as compared to a control expression level. In some embodiments, aberrant SMARCA2 expression comprises decreased SMARCA2 protein expression as compared to a control level. In some embodiments, aberrant SMARCA2 expression comprises decreased SMARCA2 mRNA expression as compared to a control level.

In some embodiments, aberrant SMARCA2 activity comprises decreased SMARCA2 activity as compared to a control activity level.

In some embodiments, the control level is a level of SMARCA2 protein expression, a level of SMARCA2 mRNA expression, a level of SMARCA2 activity or a level of SMARCA2 function in a subject or cell from a subject that does not have cancer. In some embodiments, the control level may be a level of SMARCA2 protein expression, a level of SMARCA2 mRNA expression, a level of SMARCA2 activity or a level of SMARCA2 function in a subject or cell from a subject belonging to a certain population, wherein the level is equal or about equal to the average level of protein expression, mRNA expression, activity or function of SMARCA2 observed in said population. In some embodiments, the control level may be a level of protein expression, mRNA expression, activity or function of SMARCA2 that is equal or about equal to the average level of protein expression, mRNA expression, activity or function of SMARCA2 in the population at large. In some embodiments, the control level is a level of SMARCA2 protein expression in a subject or cell from a subject that does not have cancer. In some embodiments, the control level is a level of SMARCA2 mRNA expression in a subject or cell from a subject that does not have cancer. In some embodiments, the control level is a level of SMARCA2 activity in a subject or cell from a subject that does not have cancer. In some embodiments, the control level is a level of SMARCA2 function in a subject or cell from a subject that does not have cancer.

In some aspects, a SMARCA4-targeting compound, or pharmaceutically acceptable salts or solvates thereof, can be administered orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the compound is administered orally. One skilled in the art will recognize the advantages of certain routes of administration.

In some aspects of the methods of the present disclosure, a subject has cancer. A “subject” includes a mammal. The mammal can be e.g., any mammal, e.g., a human, primate, bird, mouse, rat, fowl, dog, cat, cow, horse, goat, camel, sheep or a pig. In some embodiments, the mammal is a human.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In some aspects, the disease or condition to be treated is cancer. In other aspects, the disease or condition to be treated is a cell proliferative disorder.

As used herein, the term “responsiveness” is interchangeable with terms “responsive”, “sensitive”, and “sensitivity”, and it is meant that a subject is showing therapeutic responses when administered a composition or therapy, e.g., tumor cells or tumor tissues of the subject undergo apoptosis and/or necrosis, and/or display reduced growing, dividing, or proliferation. This term also means that a subject will or has a higher probability, relative to the population at large, of showing therapeutic responses when administered a composition or therapy e.g., tumor cells or tumor tissues of the subject undergo apoptosis and/or necrosis, and/or display reduced growing, dividing, or proliferation.

In some aspects, a “sample” can be any biological sample derived from the subject, and includes but is not limited to, cells, tissues samples, body fluids (including, but not limited to, mucus, blood, plasma, serum, urine, saliva, and semen), tumor cells, and tumor tissues. In some embodiments, the sample is selected from bone marrow, peripheral blood cells, blood, plasma and serum. Samples can be provided by the subject under treatment or testing. Alternatively, samples can be obtained by the physician according to routine practice in the art.

As used herein, a “normal cell” is a cell that cannot be classified as part of a “cell proliferative disorder”. A normal cell lacks unregulated or abnormal growth, or both, that can lead to the development of an unwanted condition or disease. In some embodiments, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “treating” or “treat” describes the management and care of a patient for the purpose of combating a disease, condition, or disorder and includes the administration of a therapy according to the methods of the present disclosure to alleviate the symptoms or complications of a disease, condition or disorder, or to eliminate the disease, condition or disorder.

Methods of the present disclosure can also be used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In some embodiments, the administration of pharmaceutical compositions leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

In some embodiments, a cancer that is to be treated is a cancer in which a member of the SWI/SNF complex, e.g., SMARCA2, is mutated, deleted, exhibits a loss of expression, exhibits a decreased in expression, and/or exhibits a loss of function (e.g., a decrease of enzymatic activity). In a non-limiting example, a cancer to be treated may be a cancer in which SMARCA2 is mutated. In a non-limiting example, a cancer to be treated may be a cancer in which the expression of SMARCA2 is decreased as compared to a control expression level (e.g. the expression level of SMARCA2 in a subject that does not have cancer). In a non-limiting example, a cancer to be treated may be a cancer in which SMARCA2 is not expressed. In a non-limiting example, a cancer to be treated may be a cancer in which the activity of SMARCA2 is decreased as compared to a control activity level (e.g. the activity level of SMARCA2 in a subject that does not have cancer).

As used herein, “parental H358” describes a wildtype NCI-H358 cell line, also referred to herein as, for example, “H358”, “NCI-H358”, and “parental”.

As used herein, “SMARCA2-knockout H358” describes a modified H358 cell line that is generated using a single expression system lentivirus (Cellecta, Inc.) containing Cas9 and sgRNA directed to SMARCA2, also referred to herein as, for example, “SMARCA2 KO”, “H358 SMARCA2 KO”, “SMARCA2-knockout NCI-H358”, and “NCI-H358 SMARCA2 KO”. In a non-limiting example, a SMARCA2-knockout H358 cell line may be a “NCI-H358 SMARCA2 KO B3” cell line, also referred to herein as, for example, “S2-B3”. In a non-limiting example, a SMARCA2-knockout H358 cell line may be a “NCI-H358 SMARCA2 KO C2” cell line, also referred to herein as, for example, “S2-C2”. In some embodiments, “SMARCA2 KO” may refer to both S2-B3 and S2-C2 cell lines.

As used herein, “SMARCA4-knockout H358” describes a modified H358 cell line that is generated using a single expression system lentivirus (Cellecta, Inc.) containing Cas9 and sgRNA directed to SMARCA4, also referred to herein as, for example, “SMARCA4 KO”, “H358 SMARCA4 KO”, “SMARCA4-knockout NCI-H358”, and “NCI-H358 SMARCA4 KO”. In a non-limiting example, a SMARCA4-knockout H358 cell line may be a “NCI-H358 SMARCA4 KO D8” cell line, also referred to herein as, for example, “S4-D8”. In a non-limiting example, a SMARCA4-knockout H358 cell line may be a “NCI-H358 SMARCA4 KO E4” cell line, also referred to herein as, for example, “S4-E4”. In some embodiments, “SMARCA4 KO” may refer to both S4-D8 and S4-E4 cell lines.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, meastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A “cell proliferative disorder of the hematologic system” is a cell proliferative disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. A hematologic cancer of the disclosure can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “cell proliferative disorder of the lung” is a cell proliferative disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Lung cancer can include malignant lung neoplasms, carcinoma in situ, typical carcinoid tumors, and atypical carcinoid tumors.

Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma,” bronchioalveolar carcinoma, giant cell carcinoma, spindle cell carcinoma, and large cell neuroendocrine carcinoma. Lung cancer can include lung neoplasms having histologic and ultrastructural heterogeneity (e.g, mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A “cell proliferative disorder of the colon” is a cell proliferative disorder involving cells of the colon. Preferably, the cell proliferative disorder of the colon is colon cancer.

Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Cell proliferative disorders of the colon can include all forms of cell proliferative disorders affecting colon cells. Cell proliferative disorders of the colon can include colon cancer, precancerous conditions of the colon, adenomatous polyps of the colon, and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ms, FAP and DCC.

A “cell proliferative disorder of the pancreas” is a cell proliferative disorder involving cells of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysaplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructural heterogeneity (e.g, mixed cell types).

A “cell proliferative disorder of the prostate” is a cell proliferative disorder involving cells of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, malignant growths or lesions of the prostate and metastatic lesions in tissue and organs in the body other than the prostate. Cell proliferative disorders of the prostate can include hyperplasia, metaplasia, and dysplasia of the prostate.

A “cell proliferative disorder of the skin” is a cell proliferative disorder involving cells of the skin. Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A “cell proliferative disorder of the ovary” is a cell proliferative disorder involving cells of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the ovary can include hyperplasia, metaplasia, and dysplasia of cells of the ovary.

A “cell proliferative disorder of the breast” is a cell proliferative disorder involving cells of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast. Breast cancer includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, MB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphocytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructural heterogeneity (e.g, mixed cell types).

In some aspects of the methods of the present disclosure, administering a compound (e.g. a SMARCA4-targeting compound) to a subject can comprise administering a pharmaceutically acceptable salt of that compound to the subject.

As used herein, “pharmaceutically acceptable salts” refer to derivatives of the compounds of the disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates), of the same salt.

### Exemplary Embodiments

Embodiment 1. A method of determining a response to at least one therapy by a subject having a cancer, wherein the at least one therapy comprises the administration of at least one SMARCA4-targeting compound, the method comprising:
a) determining a first expression level of at least one gene from at least one gene set in a biological sample collected from the subject at a first time point, wherein the first time point is prior to the administration of the at least one therapy;
b) determining a second expression level of the least one gene in a biological sample collected from the subject at a second time point, wherein the second time point is after the administration of the at least one therapy;
c) comparing the second expression level of the at least one gene to the first expression level of the at least one gene; and
d) determining that the subject is responding to the at least one therapy when the second expression level of the at least one gene is greater than the first expression level of the at least one gene.

Embodiment 2. The method of embodiment 1, wherein step (d) comprises determining that the subject is responding to the at least one therapy when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the first expression level of the at least one gene.

Embodiment 3. A method of treating a cancer in a subject, the method comprising:
a) determining a first expression level of at least one gene from at least one gene set in a biological sample collected from the subject at a first time point, wherein the first time point is prior to the administration of at least one therapeutically effective amount of at least one SMARCA4-targeting compound;
b) determining a second expression level of the least one gene in a biological sample collected from the subject at a second time point,
wherein the second time point is after the administration of at least one therapeutically effective amount of at least one SMARCA4-targeting compound;
c) comparing the second expression level of the at least one gene to the first expression level of the at least one gene; and
d) administering to the subject at least one additional therapeutically effective amount of the at least one SMARCA4-targeting compound when the second expression level of the at least one gene is greater than the first expression level of the at least one gene, or administering at least one alternative therapy to the subject when the second expression level of the at least one gene is less than the first expression level of the at least one gene.

Embodiment 4. The method of embodiment 3, wherein step (d) comprises administering to the subject at least one additional therapeutically effective amount of the at least one SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, greater than the first expression level of the at least one gene, or else administering at least one alternative therapy to the subject.

Embodiment 5. A method of determining a response to at least one therapy by a subject having a cancer, wherein the at least one therapy comprises the administration of at least one SMARCA4-targeting compound, the method comprising:
a) determining the expression level of at least one gene from at least one gene set in a biological sample collected from the subject at a first time point, wherein the first time point is after the administration of the at least one therapy;
b) comparing the expression level of the at least one gene to at least one corresponding predetermined cutoff value; and
c) determining that the subject is responding to the at least one therapy when the expression level of the at least one gene is greater than the at least one corresponding predetermined cutoff value.

Embodiment 6. The method of embodiment 5, wherein step (c) comprises determining that the subject is responding to the at least one therapy when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the at least one corresponding predetermined cutoff value.

Embodiment 7. A method of treating a cancer in a subject, wherein the subject has been previously administered at least one therapeutically effective amount of at least one SMARCA4-targeting compound, the method comprising:
a) determining the expression level of at least one gene from at least one gene set in a biological sample from the subject;
b) comparing the expression level of the at least one gene to at least one corresponding predetermined cutoff value; and
c) administering to the subject at least one additional therapeutically effective amount of the at least one SMARCA4-targeting compound when the expression level of the at least one gene is greater than the at least one corresponding predetermined cutoff value, or
administering at least one alternative therapy to the subject when the expression level of the at least one gene is less than the at least one corresponding predetermined cutoff value.

Embodiment 8. The method of embodiment 7, wherein step (c) comprises administering to the subject at least one additional therapeutically effective amount of the at least one SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the at least one corresponding predetermined cutoff value, or else administering at least one alternative therapy to the subject.

Embodiment 9. A method of determining a response to at least one therapy by a subject having a cancer, wherein the at least one therapy comprises the administration of at least one SMARCA4-targeting compound, the method comprising:
a) determining a first expression level of at least one gene from at least one gene set in a biological sample collected from the subject at a first time point, wherein the first time point is prior to the administration of the at least one therapy;
b) determining a second expression level of the least one gene in a biological sample collected from the subject at a second time point, wherein the second time point is after the administration of the at least one therapy;
c) comparing the second expression level of the at least one gene to the first expression level of the at least one gene; and
d) determining that the subject is responding to the at least one therapy when the second expression level of the at least one gene is less than the first expression level of the at least one gene.

Embodiment 10. The method of embodiment 9, wherein step (d) comprises determining that the subject is responding to the at least one therapy when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the first expression level of the at least one gene.

Embodiment 11. A method of treating a cancer in a subject, the method comprising:
a) determining a first expression level of at least one gene from at least one gene set in a biological sample collected from the subject at a first time point, wherein the first time point is prior to the administration of at least one therapeutically effective amount of at least one SMARCA4-targeting compound;
b) determining a second expression level of the least one gene in a biological sample collected from the subject at a second time point, wherein the second time point is after the administration of at least one therapeutically effective amount of at least one SMARCA4-targeting compound;
c) comparing the second expression level of the at least one gene to the first expression level of the at least one gene; and
d) administering to the subject at least one additional therapeutically effective amount of the at least one SMARCA4-targeting compound when the second expression level of the at least one gene is less than the first expression level of the at least one gene, or administering at least one alternative therapy to the subject when the second expression level of the at least one gene is greater than the first expression level of the at least one gene.

Embodiment 12. The method of embodiment 11, wherein step (d) comprises administering to the subject at least one additional therapeutically effective amount of the at least one SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the first expression level of the at least one gene, or else administering at least one alternative therapy to the subject.

Embodiment 13. A method of determining a response to at least one therapy by a subject having a cancer, wherein the at least one therapy comprises the administration of at least one SMARCA4-targeting compound, the method comprising:
a) determining the expression level of at least one gene from at least one gene set in a biological sample collected from the subject at a first time point, wherein the first time point is after the administration of the at least one therapy;
b) comparing the expression level of the at least one gene to at least one corresponding predetermined cutoff value; and
c) determining that the subject is responding to the at least one therapy when the expression level of the at least one gene is less than the at least one corresponding predetermined cutoff value.

Embodiment 14. The method of embodiment 13, wherein step (c) comprises determining that the subject is responding to the at least one therapy when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the at least one corresponding predetermined cutoff value.

Embodiment 15. A method of treating a cancer in a subject, wherein the subject has been previously administered at least one therapeutically effective amount of at least one SMARCA4-targeting compound, the method comprising:
a) determining the expression level of at least one gene from at least one gene set in a biological sample from the subject;
b) comparing the expression level of the at least one gene to at least one corresponding predetermined cutoff value; and
c) administering to the subject at least one additional therapeutically effective amount of the at least one SMARCA4-targeting compound when the expression level of the at least one gene is less than the at least one corresponding predetermined cutoff value, or administering at least one alternative therapy to the subject when the expression level of the at least one gene is greater than the at least one corresponding predetermined cutoff value.

Embodiment 16. The method of embodiment 15, wherein step (c) comprises administering to the subject at least one additional therapeutically effective amount of the at least one SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the at least one corresponding predetermined cutoff value, or else administering at least one alternative therapy to the subject.

Embodiment 17. A method of identifying at least one SMARCA4-targeting compound, the method comprising:
a) determining a first expression level of at least one gene from at least one gene set in a plurality of cells at a first time point, wherein the plurality of cells exhibits aberrant SMARCA2 expression, activity or a combination thereof;
b) treating the plurality of cells with at least one amount of at least one test compound;
c) determining a second expression level of the least one gene in the plurality of cells at a second time point;
d) comparing the second expression level of the at least one gene to the first expression level of the at least one gene; and
e) identifying the at least one test compound as a SMARCA4-targeting compound when the second expression level of the at least one gene is greater than the first expression level of the at least one gene.

Embodiment 18. The method of embodiment 17, wherein step (e) comprises identifying the at least one test compound as a SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the first expression level of the at least one gene.

Embodiment 19. A method of identifying at least one SMARCA4-targeting compound, the method comprising:
a) treating at least one cell with at least one amount of at least one test compound, wherein the at least one cell exhibits aberrant SMARCA2 expression, activity or a combination thereof;
b) determining the expression level of at least one gene from at least one gene set in the at least one cell;
c) comparing the expression level of the at least one gene to at least one corresponding predetermined cutoff value; and
d) identifying the at least one test compound as a SMARCA4-targeting compound when the expression level of the at least one gene is greater than the at least one corresponding predetermined cutoff value.

Embodiment 20. The method of embodiment 19, wherein step (d) comprises identifying the at least one test compound as a SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the at least one corresponding predetermined cutoff value.

Embodiment 21. A method of identifying at least one SMARCA4-targeting compound, the method comprising:
a) determining a first expression level of at least one gene from at least one gene set in a plurality of cells at a first time point, wherein the plurality of cells exhibits aberrant SMARCA2 expression, activity or a combination thereof;
b) treating the plurality of cells with at least one amount of at least one test compound;
c) determining a second expression level of the least one gene in the plurality of treated cells at a second time point;
d) comparing the second expression level of the at least one gene to the first expression level of the at least one gene; and
e) identifying the at least one test compound as a SMARCA4-targeting compound when the second expression level of the at least one gene is less than the first expression level of the at least one gene.

Embodiment 22. The method of embodiment 21, wherein step (e) comprises identifying the at least one test compound as a SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the first expression level of the at least one gene.

Embodiment 23. A method of identifying at least one SMARCA4-targeting compound, the method comprising:
a) treating at least one cell with at least one amount of at least one test compound, wherein the at least one cell exhibits aberrant SMARCA2 expression, activity or a combination thereof;
b) determining the expression level of at least one gene from at least one gene set in the at least treated one cell;
c) comparing the expression level of the at least one gene to at least one corresponding predetermined cutoff value; and
d) identifying the at least one test compound as a SMARCA4-targeting compound when the expression level of the at least one gene is less than the at least one corresponding predetermined cutoff value.

Embodiment 24. The method of embodiment 23, wherein step (d) comprises identifying the at least one test compound as a SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the at least one corresponding predetermined cutoff value.

Embodiment 25. The method of any one of embodiments 1, 3, 5, 7, 17 and 19, wherein the at least one gene is selected from the group consisting of the genes recited in Table 1.

Embodiment 26. The method of any one of embodiments 1, 3, 5, 7, 17 and 19, wherein the at least one gene set is selected from the gene sets recited in Table 2.

Embodiment 27. The method of any one of embodiments 9, 11, 13, 15, 21 and 23, wherein the at least one gene is selected from the group consisting of the genes recited in Table 3.

Embodiment 28. The method of any one of embodiments 9, 11, 13, 15, 21 and 23, wherein the at least one gene set is selected from the gene sets recited in Table 4.

Embodiment 29. The method of any one of the preceding embodiments, wherein the cancer exhibits aberrant SMARCA2 expression, activity, function or a combination thereof.

Embodiment 30. The method of any one of the preceding embodiments, wherein aberrant SMARCA2 expression comprises decreased SMARCA2 expression as compared to a control expression level.

Embodiment 31. The method of any one of the preceding embodiments, wherein the control expression level is the expression level of SMARCA2 in a subject that does not have cancer.

Embodiment 32. The method of any one of the preceding embodiments, wherein aberrant SMARCA2 activity comprises decreased SMARCA2 activity as compared to a control activity level.

Embodiment 33. The method of any one of the preceding embodiments, wherein the control activity level is the activity level of SMARCA2 in a subject that does not have cancer.

Embodiment 34. The method of any one of the preceding embodiments, wherein the at least one SMARCA4-targeting compound is a SMARCA4 inhibitor.

Embodiment 35. A method of modulating an epithelial/mesenchymal state in at least one cell comprising contacting the at least one cell with an effective amount of at least one SMARCA4-targeting compound.

Embodiment 36. The method of embodiment 35, wherein the SMARCA4-targeting compound is a SMARCA4 inhibitor.

Embodiment 37. The method of any one of the preceding embodiments, wherein the cell is a cancer cell.

Embodiment 38. The method of any one of the preceding embodiments, wherein the cell exhibits aberrant SMARCA2 expression, activity or a combination thereof.

Embodiment 39. The method of any one of the preceding embodiments, wherein the cell exhibits aberrant SMARCA4 expression, activity or a combination thereof.

Embodiment 40. The method of any one of embodiments 35-39, wherein modulating an epithelial/mesenchymal state in the at least one cell comprises altering the expression level of at least one gene and/or protein associated with an epithelial state.

Embodiment 41. The method of embodiment 40, wherein the at least one gene and/or protein associated with an epithelial state is E-cadherin, FOXA1 or CLDN1.

Embodiment 42. The method of any one of embodiments 35-41, wherein modulating an epithelial/mesenchymal state in the at least one cell comprises altering the expression level of at least one gene and/or protein associated with a mesenchymal state.

Embodiment 43. The method of embodiment 42, wherein the at least one gene and/or protein associated with a mesenchymal state is N-cadherin, vimentin, SNAI1 or ZEB1.

### EXAMPLES

In the following non-limiting example, SMARCA2- and SMARCA4-knockout H358 non-small cell lung cancer (NSCLC) cell lines were analyzed. The SMARCA2- and SMARCA4-knockout H358 cell lines were generated using a single expression system lentivirus (Cellecta, Inc.) containing Cas9 and sgRNA directed to SMARCA2 and SMARCA4. Briefly, the cells were plated on day zero in complete medium. 24 hours after plating, the cells were infected at multiplicity of infection (MOI) 3 in the presence of 4 μg/mL Polybrene (Millipore). Viral media was then removed 24 hours after infection. Selection using puromycin (1 μg/mL) was initiated 48 hours after infection. The infected cells were cultured under puromycin selection for 14 days. After the 14 days, the cells were diluted to single cell suspension and individual colonies were expanded. Two SMARCA2-knockout cell lines were used in the following experiments. These two SMARCA2-knockout cells lines are hereafter referred to as “S2-B3” and “S2-C2.” Two SMARCA4-knockout cell lines were used in the following experiments. These two SMARCA4-knockout cell lines are hereafter referred to as “S4-D8” and “S4-E4.” Additionally, the parental H358 cells and A549 adenocarcinomic human alveolar basal epithelial cells, hereafter referred to as “A549”, were also used in the following experiments.

### Example 1

In the following non-limiting example, the expressional profile of parental H358 cells, SMARCA2-knockout H358 cell lines and SMARCA4-knockout H358 cell lines were compared. The expression profiles of 18,559 protein coding genes in the SMARCA2- and SMARCA4-knockout cell lines were analyzed using the DriverMap Human Genome Wide Gene Expression Profiling Assay (Cellecta Inc.), which combines highly multiplexed RT-PCR amplification with Next-Generation Sequencing quantitation. Amplified cDNA products were analyzed on an Illumina NextSeq 500 sequencer using a Next Seq500/550 high Output v2 Kit (75 cycles). Read counts for each gene amplicon were normalized against endogenous housekeeping genes to enable an accurate comparison of expression levels across the series of samples. The expression DriverMap gene expression data was analyzed using GSEA software. Altered genes were compared against the Hallmark series of gene sets in the Molecular Signatures Database (MSigDB). Differences were considered significant if the false discovery rate-adjusted p-values (q-value) were less than 0.05. Using this analysis approach, the expression profiles of S2-B3, S2-C2, S4-D8 and S4-E4 cell lines were compared to the expression profile of the H358 cell line. Table 13 shows the results for 18 different Hallmark gene sets. A “+” symbol in Table 13 indicates that this gene set was upregulated in the knockout cell line (FWER p value less than 0.05). A “−” symbol in Table 13 indicates that this gene set was downregulated in the knockout cell line (FWER p value less than 0.05). Table 14 shows the top 100 genes whose expression was most significantly modulated (upregulated or downregulated) in the SMARCA2-knockout H358 cell lines. Table 15 shows the top 100 genes whose expression was most significantly different between the SMARCA2-knockout H358 cell lines and SMARCA4-knockout H358 cell lines. Table 16 shows upregulated and downregulated gene sets in the SMARCA2-knockout H358 cell lines.

TABLE 13

| | S2-B3 | S2-C2 | S4-D8 | S4-E4 |
|---|---|---|---|---|
| HALLMARK_E2F_TARGETS | + | + | + | + |
| HALLMARK_G2M_CHECKPOINT | + | + | | |
| HALLMARK_MYC_TARGETS_V1 | + | + | | |
| HALLMARK_SPERMATOGENESIS | + | + | | |
| HALLMARK_P53_PATHWAY | − | − | − | − |
| HALLMARK_TNFA_SIGNALING_VIA_NFKB | − | − | − | − |
| HALLMARK_APOPTOSIS | − | − | | |
| HALLMARK_COAGULATION | − | − | | |
| HALLMARK_COMPLEMENT | − | − | | |
| HALLMARK_EPITHELIAL_MESENCHYMAL_TRANSITION | − | − | | |
| HALLMARK_ESTROGEN_RESPONSE_EARLY | − | − | | |
| HALLMARK_HYPOXIA | − | − | | |
| HALLMARK_IL2_STAT5_SIGNALING | − | − | | |
| HALLMARK_INFLAMMATORY_RESPONSE | − | − | | |
| HALLMARK_INTERFERON_ALPHA_RESPONSE | − | − | | + |
| HALLMARK_INTERFERON_GAMMA_RESPONSE | − | − | | |
| HALLMARK_KRAS_SIGNALING_UP | − | − | | |
| HALLMARK_TGF_BETA_SIGNALING | − | − | | |

TABLE 14

| Gene | Fold | Log(Fold | | P-value |
|---|---|---|---|---|
| Symbol | Change | Change) | P-value | (adjusted) |
| CLSPN | 4.526 | 2.178 | 9.17E−27 | 1.11E−22 |
| RAD51 | 3.373 | 1.754 | 2.40E−26 | 1.11E−22 |
| MYH10 | 21.434 | 4.422 | 2.58E−26 | 1.11E−22 |
| MCM4 | 4.48 | 2.164 | 3.77E−26 | 1.22E−22 |
| ATAD2 | 3.297 | 1.721 | 2.50E−25 | 6.44E−22 |
| CCNE2 | 10.657 | 3.414 | 1.16E−24 | 2.49E−21 |
| RRM1 | 3.072 | 1.619 | 2.14E−24 | 3.80E−21 |
| MCM2 | 3.868 | 1.951 | 2.36E−24 | 3.80E−21 |
| SMARCA2 | −17.085 | −4.095 | 2.76E−24 | 3.95E−21 |
| MCM6 | 3.83 | 1.937 | 4.14E−24 | 5.34E−21 |
| SARS | −3.048 | −1.608 | 1.22E−23 | 1.43E−20 |
| RFC2 | 2.893 | 1.533 | 1.40E−23 | 1.50E−20 |
| SLFN13 | 3.381 | 1.757 | 1.56E−23 | 1.55E−20 |
| HSPA8 | 3.487 | 1.802 | 4.51E−23 | 4.16E−20 |
| PCNA | 4.431 | 2.148 | 4.93E−23 | 4.23E−20 |
| TMEM97 | 2.932 | 1.552 | 6.04E−23 | 4.86E−20 |
| E2F1 | 4.498 | 2.169 | 1.42E−22 | 1.07E−19 |
| GINS2 | 5.14 | 2.362 | 1.55E−22 | 1.11E−19 |
| BLM | 2.905 | 1.539 | 3.76E−22 | 2.55E−19 |
| SMOX | −7.348 | −2.877 | 5.66E−22 | 3.65E−19 |
| PSMD2 | 1.835 | 0.875 | 1.24E−21 | 7.59E−19 |
| NASP | 2.622 | 1.391 | 2.14E−21 | 1.26E−18 |
| WDR76 | 4.577 | 2.194 | 2.85E−21 | 1.60E−18 |
| ESCO2 | 4.083 | 2.03 | 5.51E−21 | 2.96E−18 |
| RFC5 | 2.801 | 1.486 | 6.82E−21 | 3.52E−18 |
| CDC45 | 3.297 | 1.721 | 8.48E−21 | 4.20E−18 |
| GPT2 | −2.334 | −1.223 | 9.88E−21 | 4.71E−18 |
| MCM7 | 2.51 | 1.327 | 1.20E−20 | 5.54E−18 |
| TK1 | 2.997 | 1.584 | 1.35E−20 | 5.68E−18 |
| MTHFD2 | −3.109 | −1.636 | 1.35E−20 | 5.68E−18 |
| RPA1 | 1.85 | 0.887 | 1.37E−20 | 5.68E−18 |
| FEN1 | 5.098 | 2.35 | 1.42E−20 | 5.72E−18 |
| RAD54L | 2.614 | 1.386 | 1.50E−20 | 5.77E−18 |
| ASF1B | 3.1 | 1.632 | 1.52E−20 | 5.77E−18 |
| ANLN | 2.215 | 1.147 | 1.70E−20 | 6.27E−18 |
| ACAT2 | 3.324 | 1.733 | 2.12E−20 | 7.39E−18 |
| NFIL3 | −2.023 | −1.017 | 2.12E−20 | 7.39E−18 |
| ADAP1 | −2.579 | −1.367 | 2.19E−20 | 7.42E−18 |
| IL20RB | −4.905 | −2.294 | 2.50E−20 | 8.25E−18 |
| POLA1 | 2.93 | 1.551 | 3.65E−20 | 1.18E−17 |
| FANCD2 | 2.375 | 1.248 | 3.97E−20 | 1.25E−17 |
| UHRF1 | 3.102 | 1.633 | 4.22E−20 | 1.29E−17 |
| ECT2 | 1.996 | 0.997 | 4.29E−20 | 1.29E−17 |
| FN3KRP | 2.498 | 1.321 | 4.40E−20 | 1.29E−17 |
| CDC25A | 2.872 | 1.522 | 5.01E−20 | 1.43E−17 |
| CSPG5 | 8.919 | 3.157 | 6.00E−20 | 1.68E−17 |
| HELLS | 5.301 | 2.406 | 6.25E−20 | 1.71E−17 |
| GFPT1 | −3.519 | −1.815 | 8.87E−20 | 2.38E−17 |
| ATAD5 | 3.726 | 1.898 | 9.15E−20 | 2.41E−17 |
| BRIP1 | 3.454 | 1.788 | 1.27E−19 | 3.27E−17 |
| MCM5 | 3.979 | 1.992 | 1.38E−19 | 3.48E−17 |
| PREPL | −1.705 | −0.77 | 1.73E−19 | 4.28E−17 |
| CHEK1 | 3.176 | 1.667 | 2.61E−19 | 6.34E−17 |
| CDCA7 | 2.753 | 1.461 | 3.58E−19 | 8.55E−17 |
| CDC6 | 3.02 | 1.594 | 4.11E−19 | 9.64E−17 |
| RBP1 | −37.81 | −5.241 | 4.96E−19 | 1.14E−16 |
| SLC3A2 | −2.406 | −1.267 | 9.94E−19 | 2.23E−16 |
| DTL | 3.83 | 1.937 | 1.00E−18 | 2.23E−16 |
| PCLAF | 4.202 | 2.071 | 1.02E−18 | 2.23E−16 |
| DNA2 | 2.997 | 1.584 | 1.21E−18 | 2.59E−16 |
| PPP1R15A | −3.401 | −1.766 | 1.33E−18 | 2.82E−16 |
| RPA2 | 2.082 | 1.058 | 1.61E−18 | 3.34E−16 |
| WDR34 | 2.202 | 1.139 | 1.96E−18 | 4.02E−16 |
| EXO1 | 3.397 | 1.764 | 2.01E−18 | 4.04E−16 |
| CLIP4 | −3.032 | −1.6 | 2.32E−18 | 4.60E−16 |
| NUP85 | 2.152 | 1.106 | 2.68E−18 | 5.23E−16 |
| MCM3 | 3.571 | 1.836 | 2.90E−18 | 5.58E−16 |
| UBR7 | 2.277 | 1.187 | 3.97E−18 | 7.53E−16 |
| PYGB | −3.274 | −1.711 | 4.32E−18 | 8.08E−16 |
| AK3 | −2.704 | −1.435 | 5.12E−18 | 9.44E−16 |
| WDHD1 | 2.653 | 1.408 | 5.51E−18 | 1.00E−15 |
| MAPRE3 | −1.962 | −0.972 | 6.36E−18 | 1.14E−15 |
| ORC1 | 3.529 | 1.819 | 8.05E−18 | 1.42E−15 |
| PCK2 | −4.767 | −2.253 | 8.62E−18 | 1.50E−15 |
| GABRG2 | −35.82 | −5.163 | 1.07E−17 | 1.84E−15 |
| TYMS | 4.494 | 2.168 | 1.10E−17 | 1.87E−15 |
| MCM8 | 2.285 | 1.192 | 1.22E−17 | 2.04E−15 |
| UPP1 | −5.466 | −2.45 | 1.26E−17 | 2.08E−15 |
| POLE2 | 2.818 | 1.495 | 1.48E−17 | 2.42E−15 |
| WARS | −2.974 | −1.572 | 2.06E−17 | 3.32E−15 |
| ASPM | 2.311 | 1.209 | 2.22E−17 | 3.53E−15 |
| CEBPG | −3.111 | −1.637 | 2.93E−17 | 4.61E−15 |
| MAP4K4 | −2.258 | −1.175 | 3.20E−17 | 4.96E−15 |
| SKP2 | 3.546 | 1.826 | 3.81E−17 | 5.84E−15 |
| KNTC1 | 2.606 | 1.382 | 4.99E−17 | 7.56E−15 |
| CDCA4 | 2.038 | 1.027 | 5.25E−17 | 7.86E−15 |
| LSM3 | 2.716 | 1.441 | 5.52E−17 | 8.18E−15 |
| GINS1 | 3.621 | 1.856 | 6.09E−17 | 8.93E−15 |
| TIPIN | 2.969 | 1.57 | 6.98E−17 | 1.01E−14 |
| SLC16A1 | −7.839 | −2.971 | 7.15E−17 | 1.02E−14 |
| TONSL | 3.012 | 1.591 | 8.51E−17 | 1.21E−14 |
| DSN1 | 3.561 | 1.832 | 8.81E−17 | 1.23E−14 |
| TCF19 | 3.53 | 1.82 | 8.87E−17 | 1.23E−14 |
| BRCA2 | 2.248 | 1.169 | 9.19E−17 | 1.26E−14 |
| ZNF367 | 3.895 | 1.961 | 9.91E−17 | 1.34E−14 |
| FAM105A | 3.327 | 1.734 | 1.05E−16 | 1.41E−14 |
| FANCA | 2.477 | 1.309 | 1.09E−16 | 1.45E−14 |
| RBBP7 | 1.97 | 0.978 | 1.28E−16 | 1.68E−14 |
| LRP1 | −4.816 | −2.268 | 1.29E−16 | 1.68E−14 |
| ENG | −15.627 | −3.966 | 1.35E−16 | 1.75E−14 |

TABLE 15

| Gene | Fold | Log(Fold | | P-value |
|---|---|---|---|---|
| Symbol | Change | Change) | P-value | (adjusted) |
| SFTA3 | 849.103 | 9.73 | 3.05E−41 | 3.93E−37 |
| KHDRBS2 | 163.529 | 7.353 | 8.40E−39 | 5.41E−35 |
| ATP1B1 | 4.962 | 2.311 | 1.06E−33 | 4.54E−30 |
| TMEM156 | 8.342 | 3.06 | 1.08E−32 | 3.48E−29 |
| ESPN | −17.56 | −4.134 | 2.49E−30 | 6.42E−27 |
| CYB5A | 11.178 | 3.483 | 4.20E−30 | 9.03E−27 |
| NKX2-1 | 855.192 | 9.74 | 7.40E−30 | 1.36E−26 |
| ETV4 | 3.457 | 1.789 | 1.02E−28 | 1.55E−25 |
| LFNG | −6.683 | −2.74 | 1.09E−28 | 1.55E−25 |
| SMARCA2 | −18.403 | −4.202 | 5.03E−28 | 6.48E−25 |
| IL1R1 | −10.751 | −3.426 | 7.00E−28 | 7.97E−25 |
| DBNDD2 | 4.797 | 2.262 | 7.42E−28 | 7.97E−25 |
| EDNRA | 44.644 | 5.48 | 1.17E−27 | 1.16E−24 |
| SYNPO | −29.387 | −4.877 | 1.33E−27 | 1.22E−24 |
| PLA2G4A | 3.1 | 1.632 | 2.18E−27 | 1.88E−24 |
| UCHL1 | 7.429 | 2.893 | 3.67E−27 | 2.96E−24 |
| SEL1L3 | 10.327 | 3.368 | 5.31E−27 | 4.02E−24 |
| P3H2 | −8.981 | −3.167 | 1.10E−26 | 7.85E−24 |
| LXN | −5.556 | −2.474 | 1.56E−26 | 1.06E−23 |
| SNAI1 | −10.033 | −3.327 | 6.15E−26 | 3.96E−23 |
| MMP17 | 5.008 | 2.324 | 8.33E−26 | 4.95E−23 |
| GALM | −3.582 | −1.841 | 8.45E−26 | 4.95E−23 |
| CORO1A | 4.693 | 2.23 | 1.28E−25 | 7.18E−23 |
| SH3BGRL | −1.879 | −0.91 | 2.01E−25 | 1.08E−22 |
| FOXA2 | 5.6 | 2.485 | 2.47E−25 | 1.27E−22 |
| DAB2 | −2.523 | −1.335 | 3.70E−25 | 1.83E−22 |
| CLIC3 | −14.789 | −3.886 | 4.63E−25 | 2.14E−22 |
| BCAM | −4.631 | −2.211 | 4.65E−25 | 2.14E−22 |
| ADORA2A | −12.061 | −3.592 | 7.21E−25 | 3.20E−22 |
| FAM46C | 8.945 | 3.161 | 7.70E−25 | 3.26E−22 |
| CDH6 | 408.865 | 8.675 | 7.91E−25 | 3.26E−22 |
| RAB38 | 5.49 | 2.457 | 8.08E−25 | 3.26E−22 |
| TH | −13.035 | −3.704 | 1.19E−24 | 4.64E−22 |
| NABP1 | −3.698 | −1.887 | 2.70E−24 | 1.03E−21 |
| OAT | 4.259 | 2.09 | 3.32E−24 | 1.22E−21 |
| STK32A | 120.682 | 6.915 | 3.60E−24 | 1.29E−21 |
| EGLN3 | −4.11 | −2.039 | 4.63E−24 | 1.61E−21 |
| ADGRF5 | 271.404 | 8.084 | 8.14E−24 | 2.76E−21 |
| DKK2 | 44.463 | 5.475 | 1.01E−23 | 3.35E−21 |
| FOXN3 | −3.818 | −1.933 | 1.34E−23 | 4.32E−21 |
| MCTP2 | 27.001 | 4.755 | 1.58E−23 | 4.98E−21 |
| TOX3 | 135.47 | 7.082 | 1.66E−23 | 5.09E−21 |
| LBH | −7.634 | −2.932 | 2.21E−23 | 6.61E−21 |
| PDE5A | −2.96 | −1.566 | 2.28E−23 | 6.67E−21 |
| SNTB1 | 309.095 | 8.272 | 4.34E−23 | 1.24E−20 |
| MAP4K4 | −2.644 | −1.403 | 4.88E−23 | 1.37E−20 |
| SLC16A1 | −12.318 | −3.623 | 5.38E−23 | 1.46E−20 |
| DHCR24 | 5.003 | 2.323 | 5.44E−23 | 1.46E−20 |
| MMAB | 3.015 | 1.592 | 6.62E−23 | 1.74E−20 |
| GLBIL2 | 12.698 | 3.666 | 1.14E−22 | 2.93E−20 |
| ALDH3B1 | −5.309 | −2.409 | 1.32E−22 | 3.34E−20 |
| TPP1 | −2.222 | −1.152 | 1.49E−22 | 3.69E−20 |
| PAQR7 | −4.477 | −2.162 | 1.78E−22 | 4.32E−20 |
| SLC1A5 | 2.256 | 1.174 | 2.05E−22 | 4.90E−20 |
| FIGN | −4.782 | −2.258 | 3.97E−22 | 9.29E−20 |
| FH | 2.513 | 1.329 | 4.25E−22 | 9.69E−20 |
| GLRX | 3.002 | 1.586 | 4.29E−22 | 9.69E−20 |
| SDC2 | 180.314 | 7.494 | 4.53E−22 | 1.01E−19 |
| CLIP4 | 3.129 | −1.646 | 6.65E−22 | 1.45E−19 |
| UACA | −4.396 | −2.136 | 7.65E−22 | 1.64E−19 |
| RNF141 | 3.797 | 1.925 | 1.22E−21 | 2.54E−19 |
| TSC22D1 | −3.653 | −1.869 | 1.22E−21 | 2.54E−19 |
| ME1 | 3.49 | 1.803 | 1.55E−21 | 3.17E−19 |
| SQLE | 2.394 | 1.259 | 1.60E−21 | 3.21E−19 |
| TMEM40 | 7.479 | 2.903 | 1.62E−21 | 3.21E−19 |
| OGFRL1 | 3.364 | 1.75 | 1.86E−21 | 3.63E−19 |
| TPM1 | −2.216 | −1.148 | 2.13E−21 | 4.09E−19 |
| PRICKLE1 | −5.598 | −2.485 | 2.88E−21 | 5.47E−19 |
| CTPS1 | 2.233 | 1.159 | 2.97E−21 | 5.55E−19 |
| SOCS6 | −2.564 | −1.358 | 3.03E−21 | 5.58E−19 |
| CALCOCO1 | −5.191 | −2.376 | 3.13E−21 | 5.68E−19 |
| TUBB2B | 5.102 | 2.351 | 3.63E−21 | 6.49E−19 |
| NKX1-2 | 13.398 | 3.744 | 4.50E−21 | 7.95E−19 |
| SUSD2 | −18.757 | −4.229 | 5.16E−21 | 8.99E−19 |
| INSIG1 | 3.987 | 1.995 | 5.31E−21 | 9.12E−19 |
| ENG | −19.987 | −4.321 | 6.44E−21 | 1.09E−18 |
| PSPH | 2.911 | 1.541 | 6.76E−21 | 1.13E−18 |
| ACACA | 2.253 | 1.172 | 9.53E−21 | 1.58E−18 |
| CYLD | −2.288 | −1.194 | 1.11E−20 | 1.81E−18 |
| ASAP2 | −5.982 | −2.581 | 1.16E−20 | 1.87E−18 |
| KLHL4 | −8.886 | −3.152 | 1.23E−20 | 1.95E−18 |
| FDFT1 | 2.686 | 1.425 | 1.24E−20 | 1.95E−18 |
| MVP | −2.182 | −1.126 | 1.30E−20 | 2.02E−18 |
| DUSP10 | −3.176 | −1.667 | 1.35E−20 | 2.08E−18 |
| GJB2 | −3.927 | −1.973 | 1.42E−20 | 2.15E−18 |
| TPPP3 | −24.743 | −4.629 | 1.44E−20 | 2.15E−18 |
| HBP1 | −2.149 | −1.104 | 1.48E−20 | 2.19E−18 |
| DEPTOR | 4.463 | 2.158 | 1.50E−20 | 2.19E−18 |
| GNE | −5.001 | −2.322 | 1.68E−20 | 2.43E−18 |
| TLE1 | −2.558 | −1.355 | 2.29E−20 | 3.28E−18 |
| CTSA | −2.603 | −1.38 | 2.39E−20 | 3.39E−18 |
| BTBD3 | 2.293 | 1.197 | 2.47E−20 | 3.46E−18 |
| COLCA2 | 51.184 | 5.678 | 2.52E−20 | 3.49E−18 |
| TGFB2 | −3.781 | −1.919 | 2.79E−20 | 3.80E−18 |
| PLAC8 | −34.325 | −5.101 | 2.80E−20 | 3.80E−18 |
| SHISA3 | 9.484 | 3.246 | 2.83E−20 | 3.80E−18 |
| CYP7B1 | 12.358 | 3.627 | 3.17E−20 | 4.21E−18 |
| STK39 | 3.053 | 1.61 | 4.25E−20 | 5.59E−18 |
| SLC25A11 | 2.076 | 1.054 | 4.44E−20 | 5.78E−18 |
| MYEF2 | 3.217 | 1.686 | 5.14E−20 | 6.62E−18 |

TABLE 16

| | Regulation | | | | NOM | FDR | FWER |
|---|---|---|---|---|---|---|---|
| Cell line | (Up or Down) | Gene Set | ES | NES | p-val | q-val | p-val |
| S2-B3 | Up | HALLMARK_E2F_TARGETS | −0.73 | −3.09 | 0 | 0 | 0 |
| | Up | HALLMARK_G2M_CHECKPOINT | −0.58 | −2.47 | 0 | 0 | 0 |
| | Up | HALLMARK_MYC_TARGETS_V1 | −0.37 | −1.60 | 0 | 0.008 | 0.010 |
| | Up | HALLMARK_SPERMATOGENESIS | −0.38 | −1.56 | 0 | 0.009 | 0.016 |
| | Down | HALLMARK_TNFA_SIGNALING_ | 0.63 | 2.00 | 0 | 0 | 0 |
| | | VIA_NFKB | | | | | |
| | Down | HALLMARK_EPITHELIAL_ | 0.58 | 1.85 | 0 | 0 | 0 |
| | | MESENCHYMAL_TRANSITION | | | | | |
| | Down | HALLMARK_P53_PATHWAY | 0.58 | 1.84 | 0 | 0 | 0 |
| | Down | HALLMARK_KRAS_SIGNALING_ | 0.56 | 1.78 | 0 | 4.6E−04 | 0.002 |
| | | UP | | | | | |
| | Down | HALLMARK_INTERFERON_ | 0.56 | 1.78 | 0 | 3.7E−04 | 0.002 |
| | | GAMMA_RESPONSE | | | | | |
| | Down | HALLMARK_INTERFERON_ | 0.59 | 1.77 | 0.001 | 3.1E−04 | 0.002 |
| | | ALPHA_RESPONSE | | | | | |
| | Down | HALLMARK_ESTROGEN_ | 0.56 | 1.77 | 0 | 2.6E−04 | 0.002 |
| | | RESPONSE_EARLY | | | | | |
| | Down | HALLMARK_COMPLEMENT | 0.55 | 1.74 | 0 | 3.4E−04 | 0.003 |
| | Down | HALLMARK_TGF_BETA_ | 0.62 | 1.73 | 0 | 3.0E−04 | 0.003 |
| | | SIGNALING | | | | | |
| | Down | HALLMARK_IL2_STAT5_ | 0.54 | 1.70 | 0 | 4.4E−04 | 0.005 |
| | | SIGNALING | | | | | |
| | Down | HALLMARK_INFLAMMATORY_ | 0.53 | 1.70 | 0 | 4.9E−04 | 0.006 |
| | | RESPONSE | | | | | |
| | Down | HALLMARK_UNFOLDED_ | 0.56 | 1.68 | 0 | 6.0E−04 | 0.008 |
| | | PROTEIN_RESPONSE | | | | | |
| | Down | HALLMARK_COAGULATION | 0.54 | 1.68 | 0 | 5.6E−04 | 0.008 |
| | Down | HALLMARK_HYPOXIA | 0.53 | 1.67 | 0 | 5.8E−04 | 0.009 |
| | Down | HALLMARK_APOPTOSIS | 0.52 | 1.60 | 0 | 1.7E−03 | 0.026 |
| NS2-C2 | Up | HALLMARK_E2F_TARGETS | −0.75 | −3.01 | 0 | 0 | 0 |
| | Up | HALLMARK_G2M_CHECKPOINT | −0.66 | −2.66 | 0 | 0 | 0 |
| | Up | HALLMARK_MYC_TARGETS_V1 | −0.52 | −2.08 | 0 | 0 | 0 |
| | Up | HALLMARK_SPERMATOGENESIS | −0.41 | −1.55 | 0 | 0.01184 | 0.044 |
| | Up | HALLMARK_OXIDATIVE_ | −0.38 | −1.53 | 0 | 0.01108 | 0.05 |
| | | PHOSPHORYLATION | | | | | |
| | Down | HALLMARK_TNFA_SIGNALING_ | 0.66 | 2.25 | 0 | 0 | 0 |
| | | VIA_NFKB | | | | | |
| | Down | HALLMARK_EPITHELIAL_ | 0.59 | 2.03 | 0 | 0 | 0 |
| | | MESENCHYMAL_TRANSITION | | | | | |
| | Down | HALLMARK_P53_PATHWAY | 0.58 | 1.98 | 0 | 0 | 0 |
| | Down | HALLMARK_INTERFERON_ | 0.53 | 1.83 | 0 | 0 | 0 |
| | | GAMMA_RESPONSE | | | | | |
| | Down | HALLMARK_HYPOXIA | 0.53 | 1.83 | 0 | 0 | 0 |
| | Down | HALLMARK_INTERFERON_ | 0.57 | 1.82 | 0 | 0 | 0 |
| | | ALPHA_RESPONSE | | | | | |
| | Down | HALLMARK_KRAS_SIGNALING_ | 0.52 | 1.80 | 0 | 1.43E−04 | 0.001 |
| | | UP | | | | | |
| | Down | HALLMARK_TGF_BETA_ | 0.61 | 1.77 | 0 | 1.25E−04 | 0.001 |
| | | SIGNALING | | | | | |
| | Down | HALLMARK_COAGULATION | 0.52 | 1.73 | 0 | 4.26E−04 | 0.004 |
| | Down | HALLMARK_INFLAMMATORY_ | 0.50 | 1.72 | 0 | 6.72E−04 | 0.007 |
| | | RESPONSE | | . | | | |
| | Down | HALLMARK_UV_RESPONSE_DN | 0.51 | 1.72 | 0 | 6.10E−04 | 0.007 |
| | Down | HALLMARK_IL2_STAT5_ | 0.49 | 1.70 | 0 | 8.02E−04 | 0.01 |
| | | SIGNALING | | | | | |
| | Down | HALLMARK_ESTROGEN_ | 0.49 | 1.67 | 0 | 9.68E−04 | 0.013 |
| | | RESPONSE_EARLY | | | | | |
| | Down | HALLMARK_COMPLEMENT | 0.49 | 1.66 | 0 | 0.001 | 0.017 |
| | Down | HALLMARK_APOPTOSIS | 0.49 | 1.66 | 0 | 0.001 | 0.019 |
| | Down | HALLMARK_IL6_JAK_STAT3_ | 0.52 | 1.63 | 0.001 | 0.002 | 0.029 |
| | | SIGNALING | | | | | |

Without wishing to be bound by theory, these results indicate that unique gene sets are altered upon SMARCA2 or SMARCA4 genetic knockout in H358 cells.

### Example 2

In the following non-limiting example, the expression profile of parental H358 cells, SMARCA2-knockout H358 cell lines, SMARCA4-knockout H358 cell lines and A549 cells that had been treated with a SMARCA4-targeting compound were compared. The SMARCA4-targeting compound also shows activity against SMARCA2.

To treat the cells with a SMARCA4-targeting compound, the cells were split and seeded into 10 cm during the linear/log growth phase to a final volume of 10 mL of growth media. The SMARCA4-targeting compound was diluted in DMSO and added to each culture vessel with a final DMSO concentration of 0.1%. Cells were then allowed to grow for 96 hours. At the conclusion of the treatment period, cells were harvested by centrifugation (5 minutes at 1,200 rpm) and the cell pellets were rinsed once with PBS before being frozen on dry ice until further processing and analysis.

The expression profiles of 18,559 protein coding genes in the treated cell lines were analyzed as described above in Example 1. Table 17 shows the results for 9 different Hallmark gene sets. A “+” symbol in Table 17 indicates that this gene set was upregulated by treatment with the compound (FWER p value less than 0.05). A “−” symbol in Table 17 indicates that this gene set was downregulated by treatment with the compound (FWER p value less than 0.05). Table 18 shows upregulated and downregulated gene sets in SMARCA2-knockout H358 cell lines upon 96-hour treatment with the SMARCA4-targeting compound. Table 19 shows the top 100 genes whose expression was most significantly modulated in SMARCA2-knockout H358 cell lines upon 96-hour treatment of with the SMARCA4-targeting compound. Table 20 shows the top 100 genes whose expression was most significantly different between the treated SMARCA2-knockout H358 cell line and treated SMARCA4-knockout H358 cell lines.

TABLE 17

| | | | | | | A549 | |
|---|---|---|---|---|---|---|---|
| | H358 | S2-B3 | S2-C2 | S4-D8 | S4-E4 | (24 hr) | A549 |
| HALLMARK_E2F_TARGETS | | − | | + | + | + | + |
| HALLMARK_MYC_TARGETS_V1 | | − | − | + | + | + | − |
| HALLMARK_MYC_TARGETS_V2 | | − | − | + | + | + | |
| HALLMARK_TGF_BETA_ | | + | + | | | | |
| SIGNALING | | | | | | | |
| HALLMARK_COAGULATION | | | | − | − | − | − |
| HALLMARK_EPITHELIAL_ | | | | − | − | − | |
| MESENCHYMAL_TRA | | | | | | | |
| HALLMARK_KRAS_SIGNALING_UP | | | | − | − | − | − |
| HALLMARK_INTERFERON_ALPHA_ | | − | − | − | − | − | − |
| RESPONSE | | | | | | | |
| HALLMARK_INTERFERON_ | | | − | − | − | − | − |
| GAMMA_ RESPONSE | | | | | | | |

TABLE 18

| Cell | Regulation | | | | NOM | FDR | FWER |
|---|---|---|---|---|---|---|---|
| line | (up or down) | Gene Set | ES | NES | p-val | q-val | p-val |
| S2- | Up | HALLMARK_TGF_BETA_SIGNALING | −0.48 | −1.58 | 0.011 | 0.026 | 0.03 |
| B3 | Down | HALLMARK_E2F_TARGETS | 0.68 | 2.32 | 0 | 0 | 0 |
| | Down | HALLMARK_G2M_CHECKPOINT | 0.62 | 2.12 | 0 | 0 | 0 |
| | Down | HALLMARK_MYC_TARGETS_V1 | 0.53 | 1.84 | 0 | 0.000 | 0.001 |
| | Down | HALLMARK_MTORC1_SIGNALING | 0.52 | 1.79 | 0 | 0.002 | 0.006 |
| | Down | HALLMARK_INTERFERON_ALPHA_RESPONSE | 0.55 | 1.76 | 0 | 0.001 | 0.007 |
| | Down | HALLMARK_MYC_TARGETS_V2 | 0.57 | 1.67 | 0.001 | 0.003 | 0.015 |
| S2- | Up | HALLMARK_TGF_BETA_SIGNALING | −0.51 | −1.63 | 0.003 | 0.018 | 0.025 |
| C2 | Down | HALLMARK_E2F_TARGETS | 0.74 | 2.72 | 0 | 0 | 0 |
| | Down | HALLMARK_G2M_CHECKPOINT | 0.66 | 2.41 | 0 | 0 | 0 |
| | Down | HALLMARK_MYC_TARGETS_V1 | 0.63 | 2.29 | 0 | 0 | 0 |
| | Down | HALLMARK_MTORC1_SIGNALING | 0.54 | 1.94 | 0 | 0 | 0 |
| | Down | HALLMARK_INTERFERON_ALPHA_RESPONSE | 0.55 | 1.82 | 0 | 0.001 | 0.004 |
| | Down | HALLMARK_MYC_TARGETS_V2 | 0.58 | 1.78 | 0 | 0.001 | 0.006 |
| | Down | HALLMARK_INTERFERON_GAMMA_RESPONSE | 0.45 | 1.64 | 0 | 0.006 | 0.036 |

TABLE 19

| Gene | Fold | Log(Fold | | P-value |
|---|---|---|---|---|
| Symbol | Change | Change) | P-value | (adjusted) |
| GPRC5A | 4.301 | 2.105 | 1.58E−31 | 2.04E−27 |
| ATP1B1 | −2.88 | −1.526 | 9.21E−27 | 5.94E−23 |
| PORCN | 4.81 | 2.266 | 7.18E−26 | 3.09E−22 |
| S100A11 | 2.241 | 1.164 | 4.00E−25 | 1.29E−21 |
| TMPRSS11E | 4.438 | 2.15 | 5.84E−25 | 1.50E−21 |
| GNAQ | 1.946 | 0.96 | 1.28E−24 | 2.73E−21 |
| RAB38 | −5.341 | −2.417 | 1.48E−24 | 2.73E−21 |
| CLIC3 | 13.357 | 3.74 | 1.95E−24 | 3.14E−21 |
| MYOF | 2.63 | 1.395 | 2.59E−24 | 3.44E−21 |
| UPK2 | 11.392 | 3.51 | 2.67E−24 | 3.44E−21 |
| ANXA1 | 2.238 | 1.163 | 3.06E−24 | 3.59E−21 |
| LPIN2 | −4.31 | −2.108 | 5.89E−24 | 6.33E−21 |
| SERHL2 | 19.132 | 4.258 | 6.89E−24 | 6.83E−21 |
| PSPH | −3.598 | −1.847 | 9.29E−24 | 8.55E−21 |
| STS | −4.011 | −2.004 | 1.28E−23 | 1.10E−20 |
| TPM1 | 2.46 | 1.299 | 2.36E−23 | 1.90E−20 |
| LMO7 | 2.934 | 1.553 | 4.54E−23 | 3.44E−20 |
| LYPD3 | 5.714 | 2.514 | 4.89E−23 | 3.50E−20 |
| PAQR7 | 4.691 | 2.23 | 5.78E−23 | 3.92E−20 |
| DMPK | 2.306 | 1.206 | 6.20E−23 | 4.00E−20 |
| PADI2 | 4.662 | 2.221 | 8.32E−23 | 5.03E−20 |
| LRP11 | 3.073 | 1.62 | 8.58E−23 | 5.03E−20 |
| ESPN | 5.851 | 2.549 | 2.02E−22 | 1.13E−19 |
| TBL1XR1 | −2.126 | −1.088 | 4.34E−22 | 2.33E−19 |
| SUSD2 | 22.675 | 4.503 | 5.39E−22 | 2.78E−19 |
| TRPM4 | 2.635 | 1.398 | 5.86E−22 | 2.90E−19 |
| PPL | 4.705 | 2.234 | 6.57E−22 | 3.14E−19 |
| CRIP1 | 4.216 | 2.076 | 6.89E−22 | 3.17E−19 |
| GSN | 4.922 | 2.299 | 1.85E−21 | 8.23E−19 |
| GAB1 | 2.718 | 1.443 | 2.88E−21 | 1.24E−18 |
| GCLM | −3.423 | −1.775 | 2.99E−21 | 1.24E−18 |
| ETV1 | −4.115 | −2.041 | 3.39E−21 | 1.35E−18 |
| SLC44A2 | 2.327 | 1.218 | 3.44E−21 | 1.35E−18 |
| CFI | −21.95 | −4.456 | 3.96E−21 | 1.50E−18 |
| ARF6 | 1.926 | 0.946 | 4.46E−21 | 1.64E−18 |
| SYNPO | 9.509 | 3.249 | 4.58E−21 | 1.64E−18 |
| INPP4A | 3.362 | 1.75 | 4.70E−21 | 1.64E−18 |
| EVPL | 3.073 | 1.62 | 4.88E−21 | 1.65E−18 |
| SPRED1 | −2.078 | −1.056 | 1.25E−20 | 4.13E−18 |
| FOXA2 | −3.576 | −1.838 | 1.53E−20 | 4.93E−18 |
| ABHD12 | 1.765 | 0.819 | 1.57E−20 | 4.93E−18 |
| HEG1 | −5.927 | −2.567 | 2.72E−20 | 8.35E−18 |
| STK39 | −3.089 | −1.627 | 2.96E−20 | 8.68E−18 |
| ERBB2 | 2.344 | 1.229 | 2.96E−20 | 8.68E−18 |
| IL1R1 | 4.359 | 2.124 | 3.29E−20 | 9.41E−18 |
| KDSR | −2.246 | −1.167 | 3.59E−20 | 1.01E−17 |
| CAST | 2.357 | 1.237 | 4.11E−20 | 1.13E−17 |
| TMEM120A | 2.068 | 1.048 | 5.36E−20 | 1.44E−17 |
| RELB | 2.362 | 1.24 | 6.96E−20 | 1.83E−17 |
| FLVCR2 | 6.51 | 2.703 | 7.26E−20 | 1.87E−17 |
| RAD54L | −2.114 | −1.08 | 7.87E−20 | 1.99E−17 |
| KCNK6 | 6.539 | 2.709 | 8.47E−20 | 2.10E−17 |
| KANK2 | 3.336 | 1.738 | 9.07E−20 | 2.21E−17 |
| BCAT1 | −4.914 | −2.297 | 1.07E−19 | 2.56E−17 |
| MTHFD2 | −2.373 | −1.246 | 1.57E−19 | 3.68E−17 |
| VSIR | 6.923 | 2.791 | 1.72E−19 | 3.93E−17 |
| ZNF318 | −2.768 | −1.469 | 1.74E−19 | 3.93E−17 |
| CDCA7 | −2.32 | −1.214 | 1.92E−19 | 4.26E−17 |
| CTNNB1 | −2.027 | −1.02 | 2.25E−19 | 4.92E−17 |
| VIPR1 | 8.811 | 3.139 | 3.00E−19 | 6.44E−17 |
| AJUBA | −2.391 | −1.258 | 3.15E−19 | 6.66E−17 |
| GGCT | −1.932 | −0.95 | 3.22E−19 | 6.69E−17 |
| ALCAM | −3.127 | −1.645 | 3.51E−19 | 7.19E−17 |
| CBX2 | −2.37 | −1.245 | 3.98E−19 | 8.02E−17 |
| CUL7 | 2.876 | 1.524 | 6.15E−19 | 1.22E−16 |
| PLS3 | 1.89 | 0.919 | 6.62E−19 | 1.29E−16 |
| CCNB1IP1 | −2.193 | −1.133 | 7.56E−19 | 1.45E−16 |
| BLM | −2.019 | −1.014 | 7.65E−19 | 1.45E−16 |
| ALDH3B1 | 3.665 | 1.874 | 1.01E−18 | 1.88E−16 |
| NTN1 | 3.451 | 1.787 | 1.15E−18 | 2.12E−16 |
| BCAM | 2.766 | 1.468 | 1.24E−18 | 2.24E−16 |
| MCM8 | −2.056 | −1.04 | 1.29E−18 | 2.31E−16 |
| ADGRF5 | −55.125 | −5.785 | 1.32E−18 | 2.33E−16 |
| ASMTL | 2.699 | 1.433 | 1.43E−18 | 2.50E−16 |
| BNIPL | 24.191 | 4.596 | 1.48E−18 | 2.55E−16 |
| TOMIL2 | 2.279 | 1.188 | 1.88E−18 | 3.18E−16 |
| FANCD2 | −1.881 | −0.912 | 1.92E−18 | 3.22E−16 |
| ASNS | −4.232 | −2.081 | 2.04E−18 | 3.38E−16 |
| SLC1A5 | −1.868 | −0.902 | 2.38E−18 | 3.89E−16 |
| TPP1 | 1.835 | 0.875 | 2.49E−18 | 4.01E−16 |
| CYHR1 | 2.662 | 1.412 | 2.99E−18 | 4.75E−16 |
| VSIG10 | 5.527 | 2.467 | 3.65E−18 | 5.73E−16 |
| HYAL2 | 2.24 | 1.164 | 4.09E−18 | 6.35E−16 |
| CTSA | 2.278 | 1.188 | 4.48E−18 | 6.88E−16 |
| VEGFA | −2.77 | −1.47 | 5.84E−18 | 8.86E−16 |
| TUBE1 | −3.055 | −1.611 | 5.95E−18 | 8.91E−16 |
| FAM129B | 3.018 | 1.594 | 6.10E−18 | 9.03E−16 |
| MCM6 | −2.085 | −1.06 | 6.60E−18 | 9.59E−16 |
| MPHOSPH9 | −2.136 | −1.095 | 6.62E−18 | 9.59E−16 |
| CPD | −1.843 | −0.882 | 8.01E−18 | 1.15E−15 |
| LIPH | 3.625 | 1.858 | 8.36E−18 | 1.18E−15 |
| GPRC5C | −3.218 | −1.686 | 8.61E−18 | 1.21E−15 |
| IFITM10 | 3.127 | 1.645 | 1.02E−17 | 1.41E−15 |
| HIST1H31 | −2.877 | −1.524 | 1.04E−17 | 1.43E−15 |
| USP54 | 3.055 | 1.611 | 1.20E−17 | 1.63E−15 |
| TGFBI | −6.145 | −2.619 | 1.24E−17 | 1.66E−15 |
| B4GALT4 | 2.221 | 1.151 | 1.32E−17 | 1.76E−15 |
| MMP13 | −11.099 | −3.472 | 1.64E−17 | 2.15E−15 |
| IL17RC | 2.229 | 1.156 | 1.71E−17 | 2.15E−15 |
| POC1B | −1.766 | −0.82 | 1.72E−17 | 2.15E−15 |

TABLE 20

| Gene | Fold | Log(Fold | | P-value |
|---|---|---|---|---|
| Symbol | Change | Change) | P-value | (adjusted) |
| SFTA3 | 532.044 | 9.055 | 3.73E−41 | 4.80E−37 |
| KHDRBS2 | 125.003 | 6.966 | 5.10E−39 | 3.29E−35 |
| TMPRSS1IE | 13.975 | 3.805 | 1.31E−35 | 5.64E−32 |
| HOPX | 168.615 | 7.398 | 5.81E−34 | 1.87E−30 |
| NKX2-1 | 421.343 | 8.719 | 4.01E−29 | 8.69E−26 |
| PORCN | 5.984 | 2.581 | 4.04E−29 | 8.69E−26 |
| SRPX2 | 59.623 | 5.898 | 2.18E−28 | 3.52E−25 |
| ACSL1 | 5.733 | 2.519 | 2.19E−28 | 3.52E−25 |
| SMARCA2 | −15.627 | −3.966 | 3.57E−28 | 5.12E−25 |
| NMNAT2 | 8.086 | 3.015 | 6.63E−28 | 8.55E−25 |
| FAM46C | 11.607 | 3.537 | 8.99E−28 | 1.05E−24 |
| MPP7 | 4.026 | 2.009 | 1.12E−27 | 1.20E−24 |
| STK32A | 254.89 | 7.994 | 1.26E−27 | 1.25E−24 |
| FURIN | 2.896 | 1.534 | 3.66E−27 | 3.37E−24 |
| RNF141 | 5.746 | 2.523 | 4.53E−27 | 3.89E−24 |
| PLA2G4A | 2.763 | 1.466 | 1.02E−26 | 8.18E−24 |
| CYB5A | 6.225 | 2.638 | 1.33E−26 | 1.01E−23 |
| ARHGDIB | 11.47 | 3.52 | 1.48E−26 | 1.06E−23 |
| S100A16 | 4.092 | 2.033 | 1.88E−26 | 1.27E−23 |
| LFNG | −4.69 | −2.23 | 2.18E−26 | 1.41E−23 |
| DEPTOR | 6.605 | 2.723 | 2.61E−25 | 1.55E−22 |
| SRD5A3 | 2.533 | 1.341 | 2.64E−25 | 1.55E−22 |
| GLRX | 3.487 | 1.802 | 3.28E−25 | 1.84E−22 |
| ACPP | 17.379 | 4.119 | 4.02E−25 | 2.16E−22 |
| SHROOM3 | 2.537 | 1.343 | 5.53E−25 | 2.85E−22 |
| OAT | 4.135 | 2.048 | 5.96E−25 | 2.96E−22 |
| SIX1 | −3.068 | −1.617 | 8.67E−25 | 4.14E−22 |
| SCD | 3.598 | 1.847 | 1.14E−24 | 5.24E−22 |
| ATP1B1 | 2.351 | 1.233 | 2.22E−24 | 9.74E−22 |
| SEL1L3 | 6.415 | 2.681 | 2.27E−24 | 9.74E−22 |
| SLC16A1 | −12.402 | −3.633 | 4.16E−24 | 1.73E−21 |
| ARL6IP5 | 2.273 | 1.185 | 4.44E−24 | 1.79E−21 |
| OGFRL1 | 3.79 | 1.922 | 5.28E−24 | 2.06E−21 |
| SDC2 | 220.795 | 7.787 | 9.67E−24 | 3.66E−21 |
| NET1 | 2.939 | 1.555 | 1.52E−23 | 5.61E−21 |
| ANXA2 | 2.962 | 1.566 | 1.85E−23 | 6.64E−21 |
| IL1RAP | 11.519 | 3.526 | 2.31E−23 | 7.89E−21 |
| ALDH5A1 | 3.727 | 1.898 | 2.33E−23 | 7.89E−21 |
| NCALD | 30.102 | 4.912 | 2.87E−23 | 9.47E−21 |
| ETS2 | 3.226 | 1.69 | 4.40E−23 | 1.42E−20 |
| CYLD | −2.459 | −1.298 | 4.87E−23 | 1.53E−20 |
| ADGRF1 | 4.156 | 2.055 | 5.20E−23 | 1.60E−20 |
| GABBR2 | 165.66 | 7.372 | 8.18E−23 | 2.45E−20 |
| GRHL1 | 2.312 | 1.209 | 9.24E−23 | 2.71E−20 |
| TOM1L2 | 2.765 | 1.467 | 9.74E−23 | 2.73E−20 |
| ZDHHC18 | 2.067 | 1.047 | 9.75E−23 | 2.73E−20 |
| EBP | 2.412 | 1.27 | 9.98E−23 | 2.74E−20 |
| TRAFD1 | 2.692 | 1.429 | 1.16E−22 | 3.12E−20 |
| DOPEY2 | 2.882 | 1.527 | 1.34E−22 | 3.51E−20 |
| ANK3 | 4.419 | 2.144 | 1.42E−22 | 3.67E−20 |
| MPZL2 | 3.93 | 1.975 | 1.64E−22 | 4.14E−20 |
| ESPN | −5.053 | −2.337 | 4.18E−22 | 1.02E−19 |
| NAALADL2 | 2.373 | 1.247 | 4.19E−22 | 1.02E−19 |
| GPX3 | 6.057 | 2.599 | 4.76E−22 | 1.14E−19 |
| GPRC5A | 2.138 | 1.096 | 6.13E−22 | 1.44E−19 |
| DHCR24 | 4.059 | 2.021 | 7.61E−22 | 1.75E−19 |
| STON2 | 8.829 | 3.142 | 1.24E−21 | 2.81E−19 |
| IRF2BPL | −2.779 | −1.474 | 1.76E−21 | 3.92E−19 |
| CD9 | 3.277 | 1.712 | 2.98E−21 | 6.51E−19 |
| MMAB | 2.53 | 1.339 | 3.10E−21 | 6.66E−19 |
| IDS | 3.388 | 1.761 | 3.59E−21 | 7.59E−19 |
| PTPRM | 11.948 | 3.579 | 4.87E−21 | 1.01E−18 |
| RNASET2 | 2.274 | 1.185 | 4.99E−21 | 1.02E−18 |
| SELENBP1 | 5.76 | 2.526 | 5.33E−21 | 1.07E−18 |
| RMNDSA | −2.344 | −1.229 | 7.26E−21 | 1.44E−18 |
| CYP7B1 | 11.34 | 3.503 | 1.02E−20 | 1.97E−18 |
| EDNRA | 10.043 | 3.328 | 1.03E−20 | 1.97E−18 |
| SREBF1 | 2.138 | 1.096 | 1.55E−20 | 2.94E−18 |
| ZFAND5 | −2.147 | −1.102 | 1.64E−20 | 3.06E−18 |
| CDK18 | 6.733 | 2.751 | 1.88E−20 | 3.45E−18 |
| ST3GAL5 | 5.874 | 2.554 | 1.93E−20 | 3.50E−18 |
| DKK2 | 17.834 | 4.157 | 1.96E−20 | 3.51E−18 |
| ECE1 | 3.834 | 1.939 | 2.02E−20 | 3.57E−18 |
| MYO3B | 2.96 | 1.566 | 2.19E−20 | 3.81E−18 |
| HCAR1 | 4.489 | 2.167 | 2.48E−20 | 4.26E−18 |
| SLC2A1 | 3.625 | 1.858 | 3.10E−20 | 5.26E−18 |
| DDAH1 | 2.516 | 1.331 | 4.02E−20 | 6.73E−18 |
| P2RY2 | 3.939 | 1.978 | 4.20E−20 | 6.87E−18 |
| MAPK8IP3 | 2.455 | 1.296 | 4.21E−20 | 6.87E−18 |
| ARHGAP29 | 3.511 | 1.812 | 4.69E−20 | 7.55E−18 |
| RAB6B | 4.012 | 2.004 | 5.00E−20 | 7.94E−18 |
| TRIM2 | 4.844 | 2.276 | 5.05E−20 | 7.94E−18 |
| UCHL1 | 3.314 | 1.728 | 5.33E−20 | 8.28E−18 |
| EDEM1 | 1.902 | 0.927 | 6.05E−20 | 9.29E−18 |
| RAB25 | 1.945 | 0.96 | 6.45E−20 | 9.70E−18 |
| MPZL3 | 2.839 | 1.505 | 6.47E−20 | 9.70E−18 |
| GPX1 | 4.822 | 2.27 | 8.63E−20 | 1.28E−17 |
| HS6ST2 | 3.429 | 1.778 | 8.80E−20 | 1.29E−17 |
| MPRIP | 2.511 | 1.328 | 1.10E−19 | 1.59E−17 |
| S100A13 | 2.871 | 1.521 | 1.15E−19 | 1.64E−17 |
| MGST1 | 2.141 | 1.098 | 1.32E−19 | 1.87E−17 |
| AP1S3 | 2.278 | 1.188 | 1.43E−19 | 2.00E−17 |
| CASTOR1 | 3.246 | 1.699 | 1.61E−19 | 2.23E−17 |
| FLVCR2 | 5.507 | 2.461 | 1.86E−19 | 2.54E−17 |
| SMARCA4 | 10.061 | 3.331 | 1.87E−19 | 2.54E−17 |
| DCAF16 | −1.707 | −0.772 | 1.94E−19 | 2.60E−17 |
| DBNDD2 | 2.372 | 1.246 | 2.10E−19 | 2.80E−17 |
| DPYSL2 | 6.436 | 2.686 | 2.26E−19 | 2.95E−17 |
| LUM | 39.573 | 5.306 | 2.29E−19 | 2.95E−17 |
| MYO5A | 2.031 | 1.023 | 2.29E−19 | 2.95E−17 |

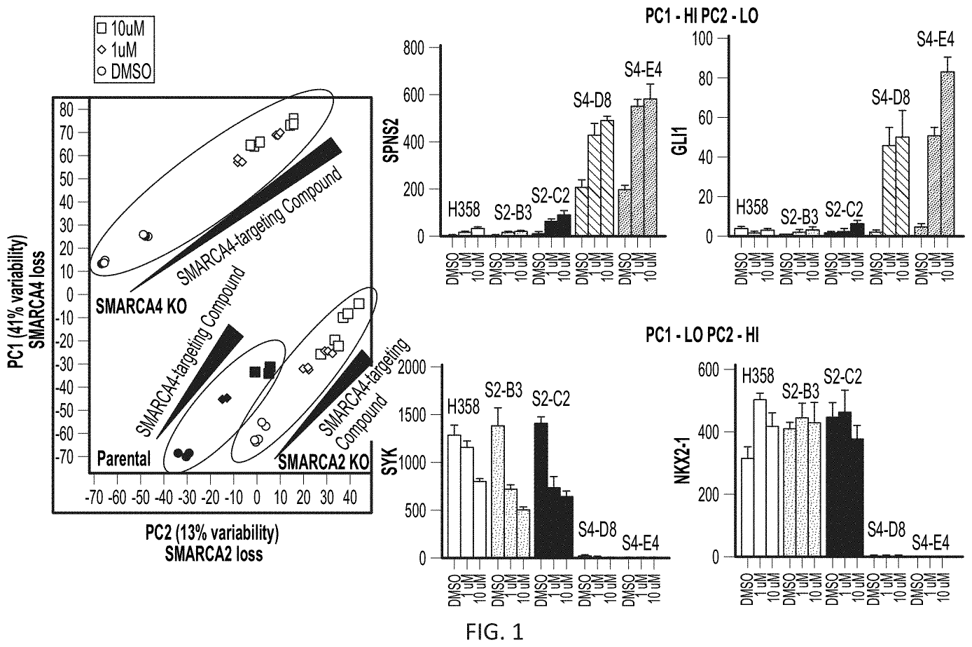

The expression profiles were also analyzed using principal component analysis to determine transcriptional changes in the treated cells. The principal component analysis (PCA) was performed by Fios Genomics using the ‘pcaMethods’ R package from BioConductor. A total of 47 samples with 12,888 features were subject to quality control evaluation, outlier detection, normalization, and then mapped onto principal components using a nonlinear iterative partial least squares algorithm. The scores of the first two PCs are plotted on the x- and y-axes of the static PCA scatterplots, respectively. FIG. 1 shows the results from this principal component analysis.

The treated cells were also analyzed by individual gene PCR. At the conclusion of the treatment period, cells were harvested, and total mRNA was extracted from the cell pellets. cDNA was synthesized and RT-PCR was performed using a TaqMan probe-system. Gene expression was normalized to the housekeeping gene, GAPDH and fold change as compared to treatment with DMSO vehicle was calculated using the DDCt method. The results of the individual gene PCR are shown in Table 21, which shows the fold change in the treated cells as compared to the vehicle treated cells.

TABLE 21

| | A549 | H358 | | | | |
|---|---|---|---|---|---|---|
| Gene | (mut) | (wt) | S2-B3 | S2-C2 | S4-D8 | S4-E4 |
| KRT80 | −47.1 | 1.2 | 1.6 | 1.6 | 1.4 | -1.9 |
| S100P | −30.8 | −5.3 | −3.1 | −4.7 | −12.5 | −7.2 |
| ARHGDIB | −9.6 | 1 | −1.7 | −2.6 | −13.9 | −10.1 |
| CLDN2 | −26 | −1.3 | 1 | −1.6 | −4.4 | −3.7 |
| SYP | 11.9 | 16.5 | 21.7 | 13.7 | 3.7 | 3.2 |
| NR4A3 | 18.8 | −1.5 | 2.5 | 4.6 | 24.7 | 20.3 |
| NR4A2 | 25.2 | 1 | 1.5 | 2.4 | 12 | 13.7 |

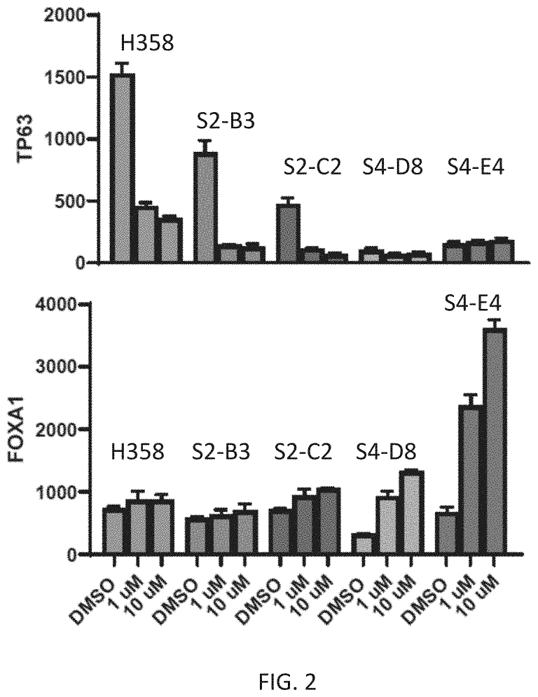

The expression levels of TP63, a transcription factor typically associated with basal characteristics, and FOXA1, a transcription factor typically associated with luminal/epithelial characteristics, were also analyzed in the treated cells. The results of this analysis are shown in FIG. 2. As shown in FIG. 2, treatment with the SMARCA4-targeting compound resulted in decreased expression of TP63 and increased expression of FOXA1 in SMARCA2-knockout cell lines.

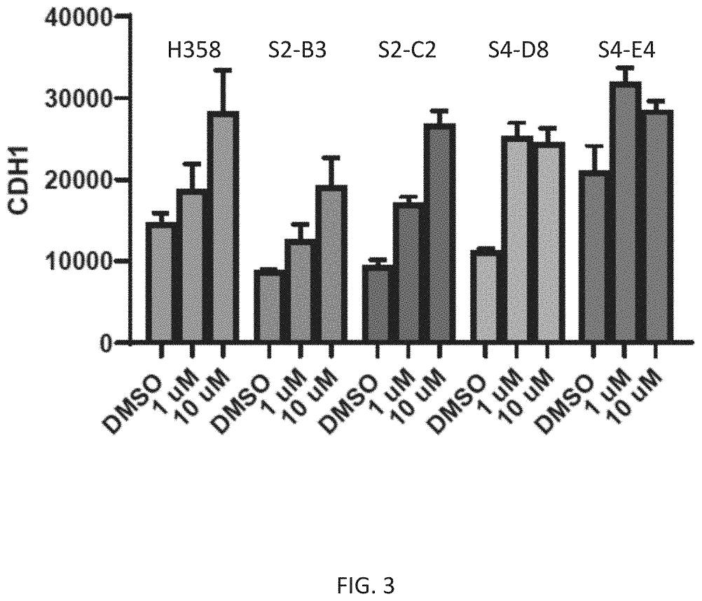

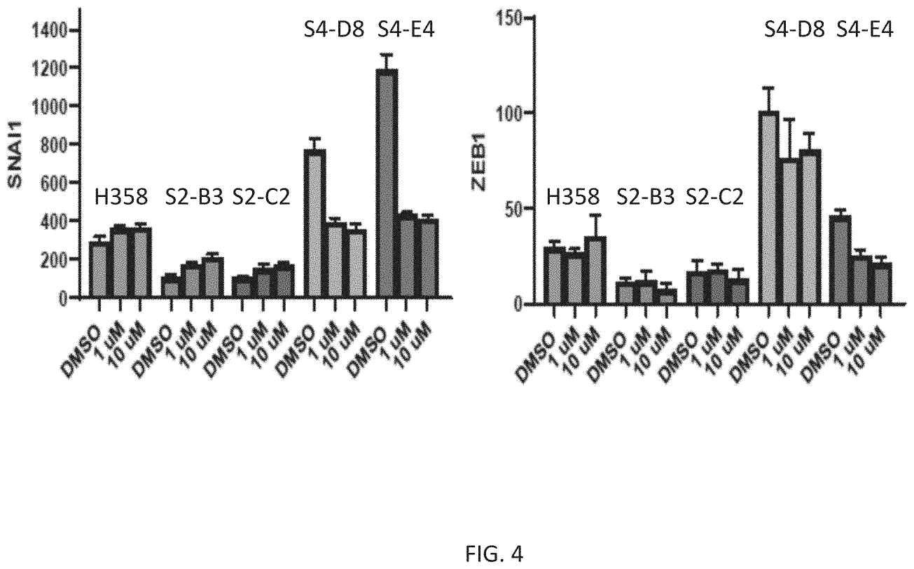

The expression levels of E-cadherin (CDH1), SNAI1 and ZEB1 were also analyzed in the treated cells. Without wishing to be bound by theory, CDH1 is commonly known as an epithelial marker, while SNAI1 and ZEB1 are commonly known as mesenchymal markers. The results of this analysis are shown in FIG. 3 and FIG. 4. As shown in FIG. 3, treatment of the SMARCA2-knockout cell lines resulted in an increase in expression of CDH1. As shown in FIG. 4, treatment of the SMARCA2-knockout cell lines resulted in no significant change in expression of SNAI1 and ZEB1.

Without wishing to be bound by theory, these results indicate that cells treated with the SMARCA4-targeting compound exhibit unique transcriptional responses as a result of the treatment. Moreover, without wishing to be bound by theory, the results also show that treatment with the SMARCA4-targeting compound resulted in changes to the cells' luminal/epithelial state.

### Example 3

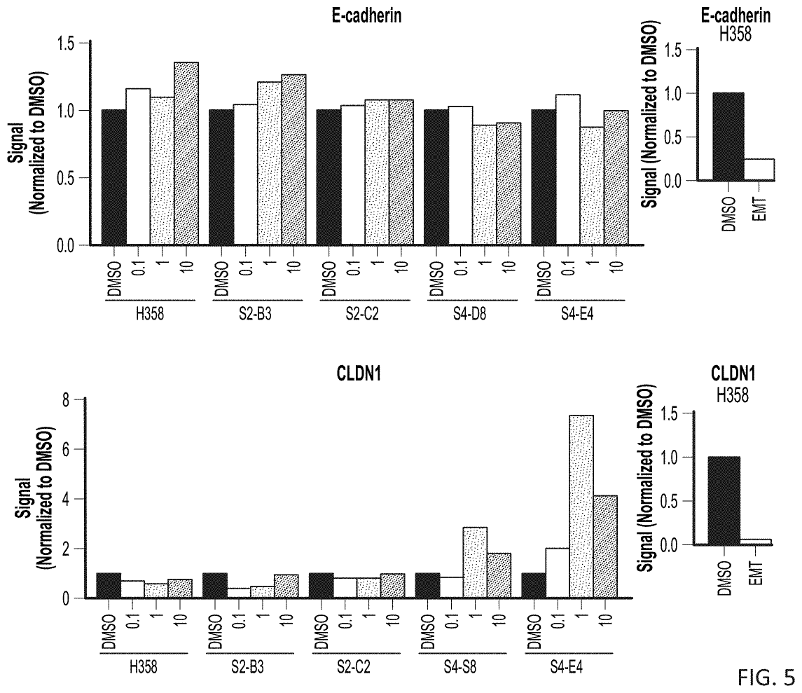

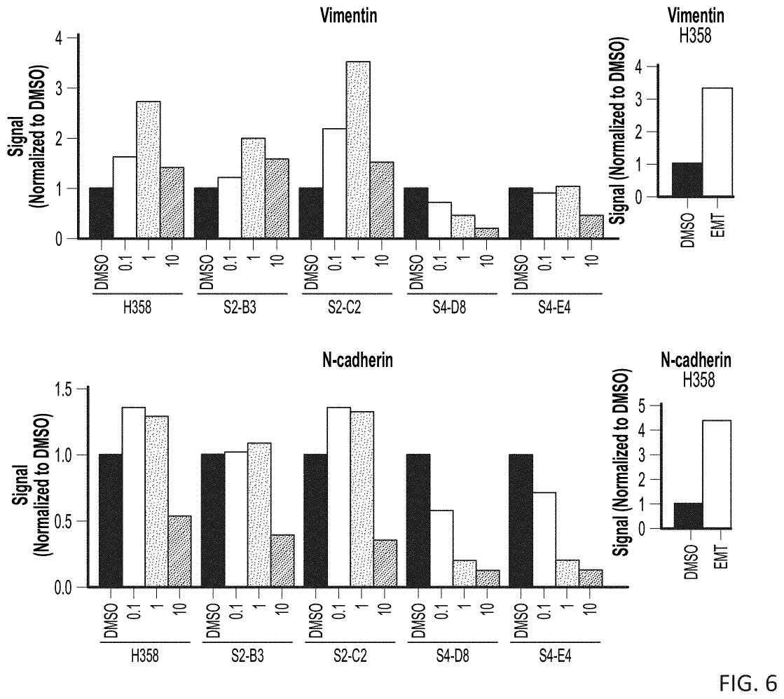

In the following non-limiting example, parental H358 cells, SMARCA2-knockout H358 cell lines and SMARCA4-knockout H358 cell lines were treated with a SMARCA4-targeting compound. The SMARCA4-targeting compound also shows activity against SMARCA2. The cells were treated either with a DMSO vehicle control, 0.1 μM of the SMARCA4-targeting compound, 1 μM of the SMARCA4-targeting compound or 10 μM of the SMARCA4-targeting compound. The treated cells were then analyzed by western blot to determine the expression of E-cadherin, CLDN1, vimentin and N-cadherin. Without wishing to be bound by theory, E-cadherin and CLDN1 are commonly known as epithelial markers while vimentin and N-cadherin are commonly known as mesenchymal markers. The results of this analysis are shown in FIG. 5 and FIG. 6. As shown in FIG. 5, treatment with the SMARCA4-targeting compound resulted in an increase in expression of CLDN1 in the SMARCA4 knockout cell lines. As shown in FIG. 6, treatment with the SMARCA4-targeting compound resulted in an increase in vimentin in SMARCA2-knockout cell lines and a decrease in N-cadherin in SMARCA2-knockout cell lines.

Without wishing to be bound by theory, these results show that treatment with the SMARCA4-targeting compound resulted in the changes to the cells' phenotype and expression of epithelial and mesenchymal markers.

### EQUIVALENTS

The foregoing description has been presented only for the purposes of illustration and is not intended to limit the disclosure to the precise form disclosed. The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

What is claimed is:

1. A method of determining a response to at least one therapy by a subject having a cancer, wherein the at least one therapy comprises the administration of at least one SMARCA4-targeting compound, the method comprising:
a) determining a first expression level of at least one gene from at least one gene set in a biological sample collected from the subject at a first time point, wherein the first time point is prior to the administration of the at least one therapy;
b) determining a second expression level of the least one gene in a biological sample collected from the subject at a second time point, wherein the second time point is after the administration of the at least one therapy;
c) comparing the second expression level of the at least one gene to the first expression level of the at least one gene; and
d) determining that the subject is responding to the at least one therapy when the second expression level of the at least one gene is greater than the first expression level of the at least one gene.

2. The method of claim 1, wherein step (d) comprises determining that the subject is responding to the at least one therapy when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the first expression level of the at least one gene.

3. A method of treating a cancer in a subject, the method comprising:
a) determining a first expression level of at least one gene from at least one gene set in a biological sample collected from the subject at a first time point,
wherein the first time point is prior to the administration of at least one therapeutically effective amount of at least one SMARCA4-targeting compound;
b) determining a second expression level of the least one gene in a biological sample collected from the subject at a second time point,
wherein the second time point is after the administration of at least one therapeutically effective amount of at least one SMARCA4-targeting compound;
c) comparing the second expression level of the at least one gene to the first expression level of the at least one gene; and
d) administering to the subject at least one additional therapeutically effective amount of the at least one SMARCA4-targeting compound when the second expression level of the at least one gene is greater than the first expression level of the at least one gene, or
administering at least one alternative therapy to the subject when the second expression level of the at least one gene is less than the first expression level of the at least one gene.

4. The method of claim 3, wherein step (d) comprises administering to the subject at least one additional therapeutically effective amount of the at least one SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times, greater than the first expression level of the at least one gene, or else administering at least one alternative therapy to the subject.

5. A method of determining a response to at least one therapy by a subject having a cancer,
wherein the at least one therapy comprises the administration of at least one SMARCA4-targeting compound, the method comprising:
a) determining the expression level of at least one gene from at least one gene set in a biological sample collected from the subject at a first time point, wherein the first time point is after the administration of the at least one therapy;
b) comparing the expression level of the at least one gene to at least one corresponding predetermined cutoff value; and
c) determining that the subject is responding to the at least one therapy when the expression level of the at least one gene is greater than the at least one corresponding predetermined cutoff value.

6. The method of claim 5, wherein step (c) comprises determining that the subject is responding to the at least one therapy when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the at least one corresponding predetermined cutoff value.

7. A method of treating a cancer in a subject,
wherein the subject has been previously administered at least one therapeutically effective amount of at least one SMARCA4-targeting compound, the method comprising:
a) determining the expression level of at least one gene from at least one gene set in a biological sample from the subject;
b) comparing the expression level of the at least one gene to at least one corresponding predetermined cutoff value; and
c) administering to the subject at least one additional therapeutically effective amount of the at least one SMARCA4-targeting compound when the expression level of the at least one gene is greater than the at least one corresponding predetermined cutoff value, or
administering at least one alternative therapy to the subject when the expression level of the at least one gene is less than the at least one corresponding predetermined cutoff value.

8. The method of claim 7, wherein step (c) comprises administering to the subject at least one additional therapeutically effective amount of the at least one SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the at least one corresponding predetermined cutoff value, or else administering at least one alternative therapy to the subject.

9. A method of determining a response to at least one therapy by a subject having a cancer,
wherein the at least one therapy comprises the administration of at least one SMARCA4-targeting compound, the method comprising:
a) determining a first expression level of at least one gene from at least one gene set in a biological sample collected from the subject at a first time point, wherein the first time point is prior to the administration of the at least one therapy;
b) determining a second expression level of the least one gene in a biological sample collected from the subject at a second time point, wherein the second time point is after the administration of the at least one therapy;
c) comparing the second expression level of the at least one gene to the first expression level of the at least one gene; and
d) determining that the subject is responding to the at least one therapy when the second expression level of the at least one gene is less than the first expression level of the at least one gene.

10. The method of claim 9, wherein step (d) comprises determining that the subject is responding to the at least one therapy when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the first expression level of the at least one gene.

11. A method of treating a cancer in a subject, the method comprising:
a) determining a first expression level of at least one gene from at least one gene set in a biological sample collected from the subject at a first time point,
wherein the first time point is prior to the administration of at least one therapeutically effective amount of at least one SMARCA4-targeting compound;
b) determining a second expression level of the least one gene in a biological sample collected from the subject at a second time point,
wherein the second time point is after the administration of at least one therapeutically effective amount of at least one SMARCA4-targeting compound;
c) comparing the second expression level of the at least one gene to the first expression level of the at least one gene; and
d) administering to the subject at least one additional therapeutically effective amount of the at least one SMARCA4-targeting compound when the second expression level of the at least one gene is less than the first expression level of the at least one gene, or administering at least one alternative therapy to the subject when the second expression level of the at least one gene is greater than the first expression level of the at least one gene.

12. The method of claim 11, wherein step (d) comprises administering to the subject at least one additional therapeutically effective amount of the at least one SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the first expression level of the at least one gene, or else administering at least one alternative therapy to the subject.

13. A method of determining a response to at least one therapy by a subject having a cancer,
wherein the at least one therapy comprises the administration of at least one SMARCA4-targeting compound, the method comprising:
a) determining the expression level of at least one gene from at least one gene set in a biological sample collected from the subject at a first time point, wherein the first time point is after the administration of the at least one therapy;
b) comparing the expression level of the at least one gene to at least one corresponding predetermined cutoff value; and
c) determining that the subject is responding to the at least one therapy when the expression level of the at least one gene is less than the at least one corresponding predetermined cutoff value.

14. The method of claim 13, wherein step (c) comprises determining that the subject is responding to the at least one therapy when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the at least one corresponding predetermined cutoff value.

15. A method of treating a cancer in a subject,
wherein the subject has been previously administered at least one therapeutically effective amount of at least one SMARCA4-targeting compound, the method comprising:
a) determining the expression level of at least one gene from at least one gene set in a biological sample from the subject;
b) comparing the expression level of the at least one gene to at least one corresponding predetermined cutoff value; and
c) administering to the subject at least one additional therapeutically effective amount of the at least one SMARCA4-targeting compound when the expression level of the at least one gene is less than the at least one corresponding predetermined cutoff value, or
administering at least one alternative therapy to the subject when the expression level of the at least one gene is greater than the at least one corresponding predetermined cutoff value.

16. The method of claim 15, wherein step (c) comprises administering to the subject at least one additional therapeutically effective amount of the at least one SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the at least one corresponding predetermined cutoff value, or else administering at least one alternative therapy to the subject.

17. A method of identifying at least one SMARCA4-targeting compound, the method comprising:
a) determining a first expression level of at least one gene from at least one gene set in a plurality of cells at a first time point, wherein the plurality of cells exhibits aberrant SMARCA2 expression, activity or a combination thereof;
b) treating the plurality of cells with at least one amount of at least one test compound;
c) determining a second expression level of the least one gene in the plurality of cells at a second time point,
d) comparing the second expression level of the at least one gene to the first expression level of the at least one gene; and
e) identifying the at least one test compound as a SMARCA4-targeting compound when the second expression level of the at least one gene is greater than the first expression level of the at least one gene.

18. The method of claim 17, wherein step (e) comprises identifying the at least one test compound as a SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the first expression level of the at least one gene.

19. A method of identifying at least one SMARCA4-targeting compound, the method comprising:
a) treating at least one cell with at least one amount of at least one test compound, wherein the at least one cell exhibits aberrant SMARCA2 expression, activity or a combination thereof;
b) determining the expression level of at least one gene from at least one gene set in the at least one cell;
c) comparing the expression level of the at least one gene to at least one corresponding predetermined cutoff value; and
d) identifying the at least one test compound as a SMARCA4-targeting compound when the expression level of the at least one gene is greater than the at least one corresponding predetermined cutoff value.

20. The method of claim 19, wherein step (d) comprises identifying the at least one test compound as a SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times greater than the at least one corresponding predetermined cutoff value.

21. A method of identifying at least one SMARCA4-targeting compound, the method comprising:
a) determining a first expression level of at least one gene from at least one gene set in a plurality of cells at a first time point, wherein the plurality of cells exhibits aberrant SMARCA2 expression, activity or a combination thereof;
b) treating the plurality of cells with at least one amount of at least one test compound;
c) determining a second expression level of the least one gene in the plurality of treated cells at a second time point;
d) comparing the second expression level of the at least one gene to the first expression level of the at least one gene; and
e) identifying the at least one test compound as a SMARCA4-targeting compound when the second expression level of the at least one gene is less than the first expression level of the at least one gene.

22. The method of claim 21, wherein step (e) comprises identifying the at least one test compound as a SMARCA4-targeting compound when the second expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the first expression level of the at least one gene.

23. A method of identifying at least one SMARCA4-targeting compound, the method comprising:
a) treating at least one cell with at least one amount of at least one test compound, wherein the at least one cell exhibits aberrant SMARCA2 expression, activity or a combination thereof;
b) determining the expression level of at least one gene from at least one gene set in the at least treated one cell;
c) comparing the expression level of the at least one gene to at least one corresponding predetermined cutoff value; and
d) identifying the at least one test compound as a SMARCA4-targeting compound when the expression level of the at least one gene is less than the at least one corresponding predetermined cutoff value.

24. The method of claim 23, wherein step (d) comprises identifying the at least one test compound as a SMARCA4-targeting compound when the expression level of the at least one gene is at least about 2 times, or at least about 3 times, or at least about 4 times, or at least about 5 times, or at least about 6 times, or at least about 7 times, or at least about 8 times or at least about 9 times, or at least about 10 times less than the at least one corresponding predetermined cutoff value.

25. The method of any one of claims 1, 3, 5, 7, 17 and 19, wherein the at least one gene is selected from the group consisting of the genes recited in Table 1.

26. The method of any one of claims 1, 3, 5, 7, 17 and 19, wherein the at least one gene set is selected from the gene sets recited in Table 2.

27. The method of any one of claims 9, 11, 13, 15, 21 and 23, wherein the at least one gene is selected from the group consisting of the genes recited in Table 3.

28. The method of any one of claims 9, 11, 13, 15, 21 and 23, wherein the at least one gene set is selected from the gene sets recited in Table 4.

29. The method of any one of the preceding claims, wherein the cancer exhibits aberrant SMARCA2 expression, activity, function or a combination thereof.

30. The method of any one of the preceding claims, wherein aberrant SMARCA2 expression comprises decreased SMARCA2 expression as compared to a control expression level.

31. The method of any one of the preceding claims, wherein the control expression level is the expression level of SMARCA2 in a subject that does not have cancer.

32. The method of any one of the preceding claims, wherein aberrant SMARCA2 activity comprises decreased SMARCA2 activity as compared to a control activity level.

33. The method of any one of the preceding claims, wherein the control activity level is the activity level of SMARCA2 in a subject that does not have cancer.

34. The method of any one of the preceding claims, wherein the at least one SMARCA4-targeting compound is a SMARCA4 inhibitor.

35. A method of modulating an epithelial/mesenchymal state in at least one cell comprising contacting the at least one cell with an effective amount of at least one SMARCA4-targeting compound.

36. The method of claim 35, wherein the SMARCA4-targeting compound is a SMARCA4 inhibitor.

37. The method of any one of the preceding claims, wherein the cell is a cancer cell.

38. The method of any one of the preceding claims, wherein the cell exhibits aberrant SMARCA2 expression, activity or a combination thereof.

39. The method of any one of the preceding claims, wherein the cell exhibits aberrant SMARCA4 expression, activity or a combination thereof.

40. The method of any one of claims 35-39, wherein modulating an epithelial/mesenchymal state in the at least one cell comprises altering the expression level of at least one gene and/or protein associated with an epithelial state.

41. The method of claim 40, wherein the at least one gene and/or protein associated with an epithelial state is E-cadherin, FOXA1 or CLDN1.

42. The method of any one of claims 35-41, wherein modulating an epithelial/mesenchymal state in the at least one cell comprises altering the expression level of at least one gene and/or protein associated with a mesenchymal state.

43. The method of claim 42, wherein the at least one gene and/or protein associated with a mesenchymal state is N-cadherin, vimentin, SNAI1 or ZEB1.
